SEQUENCE LISTING (12 sequences)

SEQ ID NO: 1 - DNA, 20 bp, Artificial Sequence
  Synthetic
  N (1)..(20): Any nucleotide
  S (1)..(20): G or C
  W (1)..(20): A or T
  misc_feature (2)..(2): n is a, c, g, or t
  misc_feature (4)..(4): n is a, c, g, or t
  misc_feature (6)..(6): n is a, c, g, or t
  misc_feature (8)..(8): n is a, c, g, or t
  misc_feature (10)..(10): n is a, c, g, or t
  misc_feature (12)..(12): n is a, c, g, or t
  misc_feature (14)..(14): n is a, c, g, or t
  misc_feature (16)..(16): n is a, c, g, or t
  misc_feature (18)..(18): n is a, c, g, or t
  misc_feature (20)..(20): n is a, c, g, or t
gnsnwnsnwnsnwnsnwnsn

SEQ ID NO: 2 - DNA, 60 bp, Artificial Sequence
  Synthetic Construct
  N (1)..(60): Any nucleotide
  S (1)..(60): G or C
  W (1)..(60): A or T
  misc_feature (20)..(20): n is a, c, g, or t
  misc_feature (22)..(22): n is a, c, g, or t
  misc_feature (24)..(24): n is a, c, g, or t
  misc_feature (26)..(26): n is a, c, g, or t
  misc_feature (28)..(28): n is a, c, g, or t
  misc_feature (30)..(30): n is a, c, g, or t
  misc_feature (32)..(32): n is a, c, g, or t
  misc_feature (34)..(34): n is a, c, g, or t
  misc_feature (36)..(36): n is a, c, g, or t
  misc_feature (38)..(38): n is a, c, g, or t
gagcctgaagacctcaccgnsnwnsnwnsnwnsnwnsngttttagcgtcttccatgcgca

SEQ ID NO: 3 - DNA, 20 bp, Artificial Sequence
  Synthetic Construct
gacatggatcgctagaaccg

SEQ ID NO: 4 - DNA, 20 bp, Artificial Sequence
  Synthetic Construct
gtcaaggtagctaagtagcg

SEQ ID NO: 5 - DNA, 20 bp, Artificial Sequence
  Synthetic Construct
gtcaagcgtgcaatggtagc

SEQ ID NO: 6 - DNA, 26 bp, Artificial Sequence
  Synthetic Construct
caccgacatggatcgctagaaccggt

SEQ ID NO: 7 - DNA, 26 bp, Artificial Sequence
  Synthetic Construct
taaaaccggttctagcgatccatgtc

SEQ ID NO: 8 - DNA, 26 bp, Artificial Sequence
  Synthetic Construct
caccgtcaaggtagctaagtagcggt

SEQ ID NO: 9 - DNA, 26 bp, Artificial Sequence
  Synthetic Construct
taaaaccgctacttagctaccttgac

SEQ ID NO: 10 - DNA, 26 bp, Artificial Sequence
  Synthetic Construct
caccgtcaagcgtgcaatggtagcgt

SEQ ID NO: 11 - DNA, 26 bp, Artificial Sequence
  Synthetic Construct
taaaacgctaccattgcacgcttgac

SEQ ID NO: 12 - DNA, 22 bp, Artificial Sequence
  Synthetic Construct
tgcgcatggaagacgctaaaac

(19) United States
(12) Patent Application Publication
(10) Pub. No.: US 2019/0169604 A1
(43) Pub. Date: Jun. 6, 2019

(54) METHODS AND COMPOSITIONS RELATED TO BARCODE ASSISTED ANCESTRAL SPECIFIC EXPRESSION (BAASE)

(71) Applicant: BOARD OF REGENTS, THE UNIVERSITY OF TEXAS SYSTEM, Austin, TX (US)

(72) Inventors: Amy BROCK, Austin, TX (US); Aziz AL'KHAFAJI, Austin, TX (US)

(21) Appl. No.: 16/324,627

(22) PCT Filed: Aug. 11, 2017
(86) PCT No.: PCT/US17/46454

§ 371 Date: Feb. 11, 2019

Related U.S. Application Data
(60) Provisional application No. 62/374,294, filed on Aug. 12, 2016.

(51) Int. Cl. C12N 15/10; C12N 15/11; C12N 9/22

(52) U.S. Cl. CPC C12N 15/1065; C12N 15/11; C12N 2740/16043; C12N 2310/20; C12N 9/22

(57) ABSTRACT

Disclosed herein are methods and platforms related to modulating expression of a gene of interest within a select population of cells comprising: providing a population of cells; providing a vehicle, plasmid, vector or recombinant virus, or equivalent thereof, capable of stably expressing a guide nucleic acid comprising randomized barcodes, thereby producing a population of barcoded cells; allowing said barcoded cell to divide, thereby forming a barcoded progeny of cells; saving an aliquot of cells; identifying the barcode in a lineage of interest from the barcoded progeny of cells; reconstituting the aliquot of saved cells, and transforming the reconstituted aliquot of cells with a transcriptional element comprising a transcriptional effector, the barcode of the lineage of interest, and a gene of interest; utilizing the transcriptional effector to modify expression of the gene of interest within the lineage of interest.

### CROSS-REFERENCE TO RELATED APPLICATIONS

This application claims benefit of U.S. Provisional Application No. 62/374,294, filed Aug. 12, 2016, incorporated herein by reference in its entirety.

### BACKGROUND

Many pathological and physiological processes, including cancer, infection, and microbiota control, are governed by the evolutionary dynamics of large heterogeneous cell populations. Tumors consist of 10⁷-10¹² cells that vary with respect to growth rate, drug response, and cell fate decisions. While rare mutations are a driving force for population adaptation, new evidence also emphasizes the contribution of epigenetic plasticity and heterogeneous cell states within clonal populations. Intratumor cell heterogeneity is a significant clinical challenge that contributes to chemoresistance and treatment failure. To inform the design of improved therapeutic strategies in cancer and infectious diseases, it is essential to develop tools for the analysis of cell heterogeneity in the context of population evolution (McGranahan et al. Cell. 2017 9; 168(4):613-628)

Recent studies have demonstrated the utility of high-diversity DNA barcode libraries in monitoring heterogeneous cell populations (Bhang et al. Nat Med, 21(5):440-8; Hata et al. Nat Med. 2016 March; 22(3):262-9; Levy et al. Nature. 2015 12; 519(7542):181-6). This is achieved by labeling each cell in a population with a unique, random, heritable sequence; lineage abundance is tracked over time by next-generation sequencing of the barcode ensemble. Changes in clonal dynamics after perturbations, such as treatment with a pharmacological agent, may reveal variation in lineage survival or proliferation rate (Bhang et al. Nat Med, 21(5):440-8; Hata et al. Nat Med. 2016 March; 22(3):262-9). This approach allows for the simultaneous observation of many cell lineage trajectories to reveal high-resolution details of population dynamics (Blundell et al. Genomics 104 (2014) 417-430). However, quantitation of lineage abundance by sequencing is a destructive measurement that limits further molecular and functional analysis of the cells in specific lineages of interest.

Currently, cell populations carrying unique heritable barcode identifiers are bulk processed for quantitation of barcode frequency by sequencing. Due to bulk processing of the cell population, lineage specific sequencing data is unattainable (Bhang et al. Nat. Med. 21, 440-448, 2015; Levy, S. F. et al. Nature 519, 181-186, 2015). Current methods for DNA sequence analysis rely upon population genome sequencing or single-cell genome sequencing. While population genome sequencing allows one to sequence with deep coverage (500-2000×), all lineage information is lost. In addition, population sequencing allows for the estimation of relative single nucleotide polymorphism (SNP) frequencies, however, this technique is unable to detect potentially important mutation frequencies below 1%. Single-cell genome sequencing allows for lineage specific genome sequences, however, with the inability to isolate clones of interest, generating significant sequencing data for lineages of interest would be prohibitively expensive and time consuming.

What is needed in the art is a method to simultaneously track lineage frequencies within a population and modulate expression of a gene(s) of interest in a lineage-specific manner.

### SUMMARY

Disclosed herein is a method of modulating expression of a gene of interest within a lineage of a select population of cells comprising: providing a population of cells; providing a vehicle, plasmid, vector or recombinant virus, or equivalent thereof, capable of stably expressing a guide nucleic acid comprising randomized barcodes, thereby producing a population of barcoded cells; allowing said barcoded cell to divide, thereby forming a barcoded progeny of cells; saving an aliquot of cells; identifying the barcode in a lineage of interest from the barcoded progeny of cells; reconstituting the aliquot of saved cells, and transforming the reconstituted aliquot of cells with a transcriptional element comprising a nucleotide guided transcriptional effector, the barcode of the lineage of interest, and a gene of interest; utilizing the transcriptional effector to modify expression of the gene of interest within the lineage of interest.

Also disclosed herein is a platform for identifying a population of cells, the platform comprising: a population of cells; a vehicle, plasmid, vector or recombinant virus, or equivalent thereof, capable of stably expressing a guide nucleic acid comprising randomized barcodes; a transcriptional element comprising a transcriptional effector, the barcode of the lineage of interest, and a gene of interest.

Further disclosed herein is a kit for use in identifying a population of cells, the kit comprising: a population of cells a vehicle, plasmid, vector or recombinant virus, or equivalent thereof, capable of stably expressing a guide nucleic acid comprising randomized barcodes; and a nucleic acid comprising a transcriptional activator, the barcode of the lineage of interest, and a gene of interest. Additional advantages will be set forth in part in the description that follows or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1A-D shows lineage-specific expression of GFP. (A) Generation and lineage specific gene activation of independent barcoded gRNA populations. Three different barcodes were randomly generated following the GNSNWNSNWNSNWNSNWNSN (SEQ ID NO: 1) template and assembled into lentiviral gRNA expression cassettes. Cell lines: HEK 293T, Caco2, and MDA-MB-231 were independently transduced with the three different barcode gRNAs and selected for stable integration. The barcoded populations were then co-transfected with each of one of the Recall plasmids and the dCas9-VPR plasmid. GFP expression was assessed 48 h post transfection via flow cytometry. (B) View of the lineage specific expression components. The base Recall Plasmid contains a Golden Gate multiple cloning site for modular assembly of the 3× Barcode+PAM array and adjacent downstream miniCMV promoter+sfGFP gene within the Recall plasmid. In the presence of the matching barcode gRNA/dCas9-VPR complex, binding of the barcode arrays by the transcriptopnal activator dCas9-VPR will drive expression of sfGFP. In the case of mismatching barcode gRNA/dCas9-VPR complex, binding of the barcode arrays will not occur and expression of sfGFP will not be driven. (C) Overlaid histograms comparing high GFP expression for instances of matching barcode gRNA/Recall plasmid and nominal expression for instances of mismatch. GFP expression was measured via flow cytometry. (D) Error load graphs showing percent positive population activation at a given error rate.

FIG. 2 shows isolation and manipulation of a single lineage of interest from high diversity population. High diversity gRNA barcoded HEK 293T cell population was generated with a GNSNWNSNWNSNWNSNWNSN (SEQ ID NO: 1) template. The HEK 293T Bg-A population was spiked in with the high diversity population to obtain a 1% and 0.1% Bg-A mixed population. Bg-A cells were then isolated from the mixed population via co-transfection of the Recall A plasmid and dCas9-VPR plasmid and FACS based off of GFP expression, (b) sequencing confirmation of barcode and surrounding sequence, (c) bi-directional lineage specific gene expression of BAX and sfGFP. (d) GFP activation in cells of the Bax-activated cell lineage. Arrowheads indicate example cells that activate the reporter and complete apoptosis over approximately 20 h.

FIG. 3 demonstrates lineage specific activation of a reporter gene and confirms the relationship between reporter activation and expression of the transcriptional activator. Populations of HEK 293T cells stably expressing either barcode-gRNA_1 (KM1) or barcode-gRNA_2 (KM2) were transfected via lipofectamine 3000 with both Recall Plasmid_1 and dCas9-VPR plasmid. Populations A(1-3) denote KM1 and B(1-3) KM2 barcoded cells. Populations A1 and B1 were transfected with 15 ng of Recall Plasmid_1 and no dCas9-VPR plasmid. Both populations display minimal increase in fluorescent cells per image post transfection, underscoring the necessity of the transcriptional activator, dCas9-VPR, to drive expression of sfGFP. The KM1 populations, A2 and A3, were transfected with 15 ng Recall Plasmid_1 and 300 ng and 900 ng of dCas9-VPR plasmid respectively. Populations A2 and A3 display a rapid increase in fluorescent cells per image post transfection, with increased signal coming from increased concentrations of dCas9-VPR. As the expressed barcode gRNA_1 of the KM1 cell line is a match for the barcode site on Recall Plasmid_1, the gRNA_1 can complex with dCas9-VPR, forming a targeting complex for expression of sfGFP on the Recall Plasmid_1. The KM2 populations, B2 and B3, were transfected with 15 ng Recall Plasmid_1 and 300 ng and 900 ng of dCas9-VPR plasmid respectively. Populations B2 and B3 display a minimal increase in fluorescent cells per image post transfection. As the expressed barcode gRNA_2 of the KM2 cell line is a mismatch for the barcode site on Recall Plasmid_1, the gRNA_2/dCas9-VPR complex is not a targeting complex for expression of sfGFP on the Recall Plasmid_1. Fluorescent cells per image were quantified using the IncuCyte live cell analysis system over 68 hours at two-hour intervals. Nine images were taken per well.

FIG. 4 shows successful lineage specific activation of a reporter gene and demonstrates that activation increases with amount of guide nucleotide sequence. Populations of HEK 293T cells stably expressing either barcode-gRNA_1 (KM1) or barcode-gRNA_2 (KM2) were transfected via lipofectamine 3000 with both Recall Plasmid_1 and dCas9-VPR plasmid. Populations A(1-2) denote KM1 and B(1-2) KM2 barcoded cells. The KM1 populations, A1 and A2, were transfected with 300 ng dCas9-VPR plasmid and 15 ng and 30 ng of Recall Plasmid_1 respectively. Populations A1 and A2 display a rapid increase in fluorescent cells per image post transfection, with increased signal coming from increased concentrations of Recall Plasmid_1. The KM2 populations, B1 and B2, were transfected with 300 ng dCas9-VPR plasmid and 15 ng and 30 ng of Recall Plasmid_1 respectively. Populations B1 and B2 display a minimal increase in fluorescent cells per image post transfection, with slightly increased background signal coming from increased concentrations of Recall Plasmid_1.

FIGS. 5A and 5B shows recall plasmid schematics. Shown is a plasmid chassis that contains multiple TIIS cloning site for the fascicle introduction of barcode landing pads and gene(s) of interest to be expressed.

FIG. 6 shows a recall plasmid containing miniCMV-sfGFP. Primed for lineage specific gene expression of sfGFP, lineage of interest barcode+PAM sequence can be introduced in the BbsI cloning site.

FIG. 7 shows a recall plasmid containing 3× Barcode_A-miniCMV-sfGFP. Primed for lineage specific gene expression of sfGFP in cells containing the expressed barcode gRNA_A (GACATGGATCGCTAGAACCG, SEQ ID NO: 3).

FIG. 8 shows recall plasmid containing miniCMV-BAX-3× Barcode_A-miniCMV-sfGFP. Primed for lineage specific bi-directional gene expression of BAX and sfGFP in cells containing the expressed barcode gRNA_A (GACATGGATCGCTAGAACCG, SEQ ID NO: 3).

FIG. 9A-B shows Bg-A landing pad array assembly. The 3× barcode landing pad arrays were assembled by first annealing complimentary oligonucleotides containing the barcode of interests and PAM site along with the specified overhangs A-F (a). When combined, these specified overhangs drive assembly of the individual double stranded barcodes to both make the 3× barcode array as well as direct integration into the BbsI digested Recall plasmid (b). Similar schemes were used to assemble larger barcode arrays.

FIG. 10A-C shows lineage specific gene activation efficiency of 1×, 3×, 6× barcode landing pads at different concentrations of dCas9-VPR. Time lapse fluorescent analysis of percent green object confluence of HEK293 Ts Bg-A and Bg-B populations co-transfected with dCas9-VPR and 80 ng of Recall-A_GFP plasmids with a 1×, 3×, or 6× barcode array in a 24 well plate. These graphs compare recall activation efficiency between Recall-A_GFP plasmids with a 1×, 3×, or 6× barcode array at given dCas9-VPR amounts.

FIG. 11A-C shows lineage specific gene activation efficiency with increase concentrations of dCas9-VPR in coordination with 1×, 3×, or 6× barcode landing pads. Time lapse fluorescent analysis of percent green object confluence of HEK293 Ts Bg-A and Bg-B populations co-transfected with 0, 100, 300, and 900 ng of dCas9-VPR and 80 ng of Recall-A_GFP plasmids with a 1×, 3×, or 6× barcode array in a 24 well plate. These graphs compare recall activation efficiency of increasing amounts dCas9-VPR when co-transfected with 80 ng Recall-A_GFP plasmids with a 1×, 3×, or 6× barcode array.

### DETAILED DESCRIPTION

The methods and platform described herein may be understood more readily by reference to the following detailed description of specific aspects of the disclosed subject matter and the Examples included therein.

Before the present methods and platform are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid the reader in distinguishing the various components, features, or steps of the disclosed subject matter. The identifiers “first” and “second” are not intended to imply any particular order, amount, preference, or importance to the components or steps modified by these terms.

By convention, polynucleotides that are formed by 3′-5′ phosphodiester linkages (including naturally occurring polynucleotides) are said to have 5′-ends and 3′-ends because the nucleotide monomers that are incorporated into the polymer are joined in such a manner that the 5′ phosphate of one mononucleotide pentose ring is attached to the 3′ oxygen (hydroxyl) of its neighbor in one direction via the phosphodiester linkage. Thus, the 5′-end of a polynucleotide molecule generally has a free phosphate group at the 5′ position of the pentose ring of the nucleotide, while the 3′ end of the polynucleotide molecule has a free hydroxyl group at the 3′ position of the pentose ring. Within a polynucleotide molecule, a position that is oriented 5′ relative to another position is said to be located “upstream,” while a position that is 3′ to another position is said to be “downstream.” This terminology reflects the fact that polymerases proceed and extend a polynucleotide chain in a 5′ to 3′ fashion along the template strand. Also included are bidirectional nucleic acids, in which a promoter activates a molecule in one direction and another molecule in the opposite direction. Unless denoted otherwise, whenever a polynucleotide sequence is represented, it will be understood that the nucleotides are in 5′ to 3′ orientation from left to right.

As used herein, it is not intended that the term “polynucleotide” be limited to naturally occurring polynucleotide structures, naturally occurring nucleotides sequences, naturally occurring backbones or naturally occurring internucleotide linkages. One familiar with the art knows well the wide variety of polynucleotide analogues, unnatural nucleotides, non-natural phosphodiester bond linkages and internucleotide analogs that find use with the invention.

As used herein, the expressions “nucleotide sequence,” “sequence of a polynucleotide,” “nucleic acid sequence,” “polynucleotide sequence”, and equivalent or similar phrases refer to the order of nucleotide monomers in the nucleotide polymer. By convention, a nucleotide sequence is typically written in the 5′ to 3′ direction. Unless otherwise indicated, a particular polynucleotide sequence of the invention optionally encompasses complementary sequences, in addition to the sequence explicitly indicated.

The term “guide nucleotide” refers to a synthetic nucleotide sequence, such as RNA (referred to as “guide RNA” or “gRNA”), consisting of a binding site for DNA binding proteins, such as Cas9, and a specific nucleotide targeting sequence.

As used herein, the term “gene” generally refers to a combination of polynucleotide elements, that when operatively linked in either a native or recombinant manner, provide some product or function. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” encompasses mRNA, cDNA and genomic forms of a gene.

In some aspects, the genomic form or genomic clone of a gene includes the sequences of the transcribed mRNA, as well as other non-transcribed sequences which lie outside of the transcript. The regulatory regions which lie outside the mRNA transcription unit are termed 5′ or 3′ flanking sequences. A functional genomic form of a gene typically contains regulatory elements necessary, and sometimes sufficient, for the regulation of transcription. The term “promoter” is generally used to describe a DNA region, typically but not exclusively 5′ of the site of transcription initiation, sufficient to confer accurate transcription initiation. In some aspects, a “promoter” also includes other cis-acting regulatory elements that are necessary for strong or elevated levels of transcription, or confer inducible transcription. In some embodiments, a promoter is constitutively active, while in alternative embodiments, the promoter is conditionally active (e.g., where transcription is initiated only under certain physiological conditions).

Generally, the term “regulatory element” refers to any cis-acting genetic element that controls some aspect of the expression of nucleic acid sequences. In some uses, the term “promoter” comprises essentially the minimal sequences required to initiate transcription. In some uses, the term “promoter” includes the sequences to start transcription, and in addition, also include sequences that can upregulate or downregulate transcription, commonly termed “enhancer elements” and “repressor elements,” respectively.

Specific DNA regulatory elements, including promoters and enhancers, generally only function within a class of organisms. For example, regulatory elements from the bacterial genome generally do not function in eukaryotic organisms. However, regulatory elements from more closely related organisms frequently show cross functionality. For example, DNA regulatory elements from a particular mammalian organism, such as human, will most often function in other mammalian species, such as mouse. Furthermore, in designing recombinant genes that will function across many species, there are consensus sequences for many types of regulatory elements that are known to function across species, e.g., in all mammalian cells, including mouse host cells and human host cells.

As used herein, the expressions “in operable combination,” “in operable order,” “operatively linked,” “operatively joined” and similar phrases, when used in reference to nucleic acids, refer to the operational linkage of nucleic acid sequences placed in functional relationships with each other. For example, an operatively linked promoter, enhancer elements, open reading frame, 5′ and 3′ UTR, and terminator sequences result in the accurate production of an RNA molecule. In some aspects, operatively linked nucleic acid elements result in the transcription of an open reading frame and ultimately the production of a polypeptide (i.e., expression of the open reading frame).

As used herein, the term “genome” refers to the total genetic information or hereditary material possessed by an organism (including viruses), i.e., the entire genetic complement of an organism or virus. The genome generally refers to all of the genetic material in an organism's chromosome(s), and in addition, extra-chromosomal genetic information that is stably transmitted to daughter cells (e.g., the mitochondrial genome). A genome can comprise RNA or DNA. A genome can be linear (mammals) or circular (bacterial). The genomic material typically resides on discrete units such as the chromosomes.

As used herein, a “polypeptide” is any polymer of amino acids (natural or unnatural, or a combination thereof), of any length, typically but not exclusively joined by covalent peptide bonds. A polypeptide can be from any source, e.g., a naturally occurring polypeptide, a polypeptide produced by recombinant molecular genetic techniques, a polypeptide from a cell, or a polypeptide produced enzymatically in a cell-free system. A polypeptide can also be produced using chemical (non-enzymatic) synthesis methods. A polypeptide is characterized by the amino acid sequence in the polymer. As used herein, the term “protein” is synonymous with polypeptide. The term “peptide” typically refers to a small polypeptide, and typically is smaller than a protein. Unless otherwise stated, it is not intended that a polypeptide be limited by possessing or not possessing any particular biological activity.

As used herein, the expressions “codon utilization” or “codon bias” or “preferred codon utilization” or the like refers, in one aspect, to differences in the frequency of occurrence of any one codon from among the synonymous codons that encode for a single amino acid in protein-coding DNA (where many amino acids have the capacity to be encoded by more than one codon). In another aspect, “codon use bias” can also refer to differences between two species in the codon biases that each species shows. Different organisms often show different codon biases, where preferences for which codons from among the synonymous codons are favored in that organism's coding sequences.

As used herein, the terms “vector,” “vehicle,” “construct” and “plasmid” are used in reference to any recombinant polynucleotide molecule that can be propagated and used to transfer nucleic acid segment(s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contain operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

As used herein, the term “host cell” refers to any cell that contains a heterologous nucleic acid. The heterologous nucleic acid can be a vector, such as a shuttle vector or an expression vector. In some aspects, the host cell is able to drive the expression of genes that are encoded on the vector. In some aspects, the host cell supports the replication and propagation of the vector. Host cells can be bacterial cells such as E. coli, or mammalian cells (e.g., human cells or mouse cells). When a suitable host cell (such as a suitable mouse cell) is used to create a stably integrated cell line, that cell line can be used to create a complete transgenic organism.

Methods (i.e., means) for delivering vectors/constructs or other nucleic acids (such as in vitro transcribed RNA) into host cells such as bacterial cells and mammalian cells are well known to one of ordinary skill in the art, and are not provided in detail herein. Any method for nucleic acid delivery into a host cell finds use with the invention.

For example, methods for delivering vectors or other nucleic acid molecules into bacterial cells (termed transformation) such as Escherichia coli are routine, and include electroporation methods and transformation of E. coli cells that have been rendered competent by previous treatment with divalent cations such as CaCl₂.

Methods for delivering vectors or other nucleic acid (such as RNA) into mammalian cells in culture (termed transfection) are routine, and a number of transfection methods find use with the invention. These include but are not limited to calcium phosphate precipitation, electroporation, lipid-based methods (liposomes or lipoplexes) such as Transfectamine® (Life Technologies™) and TransFectin™ (Bio-Rad Laboratories), cationic polymer transfections, for example using DEAE-dextran, direct nucleic acid injection, biolistic particle injection, and viral transduction using engineered viral carriers (termed transduction, using e.g., engineered herpes simplex virus, adenovirus, adeno-associated virus, vaccinia virus, Sindbis virus), and sonoporation. Any of these methods find use with the invention.

As used herein, the term “recombinant” in reference to a nucleic acid or polypeptide indicates that the material (e.g., a recombinant nucleic acid, gene, polynucleotide, polypeptide, etc.) has been altered by human intervention. Generally, the arrangement of parts of a recombinant molecule is not a native configuration, or the primary sequence of the recombinant polynucleotide or polypeptide has in some way been manipulated. A naturally occurring nucleotide sequence becomes a recombinant polynucleotide if it is removed from the native location from which it originated (e.g., a chromosome), or if it is transcribed from a recombinant DNA construct. A gene open reading frame is a recombinant molecule if that nucleotide sequence has been removed from it natural context and cloned into any type of nucleic acid vector (even if that ORF has the same nucleotide sequence as the naturally occurring gene). Protocols and reagents to produce recombinant molecules, especially recombinant nucleic acids, are well known to one of ordinary skill in the art. In some embodiments, the term “recombinant cell line” refers to any cell line containing a recombinant nucleic acid, that is to say, a nucleic acid that is not native to that host cell.

As used herein, the terms “heterologous” or “exogenous” as applied to polynucleotides or polypeptides refers to molecules that have been rearranged or artificially supplied to a biological system and are not in a native configuration (e.g., with respect to sequence, genomic position or arrangement of parts) or are not native to that particular biological system. These terms indicate that the relevant material originated from a source other than the naturally occurring source, or refers to molecules having a non-natural configuration, genetic location or arrangement of parts. The terms “exogenous” and “heterologous” are sometimes used interchangeably with “recombinant.”

As used herein, the terms “native” or “endogenous” refer to molecules that are found in a naturally occurring biological system, cell, tissue, species or chromosome under study. A “native” or “endogenous” gene is a generally a gene that does not include nucleotide sequences other than nucleotide sequences with which it is normally associated in nature (e.g., a nuclear chromosome, mitochondrial chromosome or chloroplast chromosome). An endogenous gene, transcript or polypeptide is encoded by its natural locus, and is not artificially supplied to the cell.

As used herein, the expression “homologous recombination” refers to a genetic process in which nucleotide sequences are exchanged between two similar molecules of DNA. Homologous recombination (HR) is used by cells to accurately repair harmful breaks that occur on both strands of DNA, known as double-strand breaks or other breaks that generate overhanging sequences. Various molecular events are thought to control HR; however, an understanding of the molecular mechanisms underlying HR are not required to make and use the invention. After some types of DNA damage, various forms of HR repair the damage using the following general steps: (i) resection or excision of the damaged DNA; (ii) strand invasion where an end of the broken DNA molecule “invades” a similar or identical DNA molecule in a region of homology that is not damaged; (iii) finally, either of two pathways is used to effectuate the repair, involving DNA synthesis and relegation. HR requires that there be present some identical or homologous strand of DNA that serves as a template to direct the repair of the damaged DNA.

As used herein, the expressions “donor polynucleotide” or “donor fragment” or “template DNA” refer to the strand of DNA that is the recipient strand during HR strand invasion that is initiated by the damaged DNA. The donor polynucleotide serves as template material to direct the repair of the damaged DNA region.

As used herein, the expression “non-homologous end joining (NHEJ)” refers to a cellular pathway that repairs double-strand breaks in DNA. NHEJ is referred to as “non-homologous” DNA repair because the break ends are directly ligated to each other without the need for a homologous template, in contrast to homologous recombination, which requires a homologous sequence to guide the repair. NHEJ frequently results in imprecise DNA repair, and can introduce errors (including deletions and insertions) in the repaired DNA.

As used herein, the term “marker” most generally refers to a biological feature or trait that, when present in a cell (e.g., is expressed), results in an attribute or phenotype that visualizes or identifies the cell as containing that marker. As used herein, the expressions “selectable marker” or “screening marker” or “positive selection marker” refer to a marker that, when present in a cell, results in an attribute or phenotype that allows selection or segregated of those cells from other cells that do not express the selectable marker trait. A variety of genes are used as selectable markers, e.g., genes encoding drug resistance or auxotrophic rescue are widely known. For example, kanamycin (neomycin) resistance can be used as a trait to select bacteria that have taken up a plasmid carrying a gene encoding for bacterial kanamycin resistance (e.g., the enzyme neomycin phosphotransferase II). Non-transfected cells will eventually die off when the culture is treated with neomycin or similar antibiotic.

A similar mechanism can also be used to select for transfected mammalian cells containing a vector carrying a gene encoding for neomycin resistance (either one of two aminoglycoside phosphotransferase genes; the neo selectable marker). This selection process can be used to establish stably transfected mammalian cell lines. Geneticin (G418) is commonly used to select the mammalian cells that contain stably integrated copies of the transfected genetic material.

As used herein, the expressions “negative selection” or “negative screening marker” refers to a marker that, when present (e.g., expressed, activated, or the like) allows identification of a cell that does not comprise a selected property or trait (e.g., as compared to a cell that does possess the property or trait).

A wide variety of positive and negative selectable markers are known for use in prokaryotes and eukaryotes, and selectable marker tools for plasmid selection in bacteria and mammalian cells are widely available. Bacterial selection systems include, for example but not limited to, ampicillin resistance (β-lactamase), chloramphenicol resistance, kanamycin resistance (aminoglycoside phosphotransferases), and tetracycline resistance. Mammalian selectable marker systems include, for example but not limited to, neomycin/G418 (neomycin phosphotransferase II), methotrexate resistance (dihydropholate reductase; DHFR), hygromycin-B resistance (hygromycin-B phosphotransferase), and blasticidin resistance (blasticidin S deaminase).

As used herein, the term “reporter” refers generally to a moiety, chemical compound or other component that can be used to visualize, quantitate or identify desired components of a system of interest. Reporters are commonly, but not exclusively, genes that encode reporter proteins. For example, a “reporter gene” is a gene that, when expressed in a cell, allows visualization or identification of that cell, or permits quantitation of expression of a recombinant gene. For example, a reporter gene can encode a protein, for example, an enzyme whose activity can be quantitated, for example, chloramphenicol acetyltransferase (CAT) or firefly luciferase protein. Reporters also include fluorescent proteins, for example, green fluorescent protein (GFP) or any of the recombinant variants of GFP, including enhanced GFP (EGFP), blue fluorescent proteins (BFP and derivatives), cyan fluorescent protein (CFP and other derivatives), yellow fluorescent protein (YFP and other derivatives) and red fluorescent protein (RFP and other derivatives).

As used herein, the term “tag” as used in protein tags refers generally to peptide sequences that are genetically fused to other protein open reading frames, thereby producing recombinant fusion proteins. Ideally, the fused tag does not interfere with the native biological activity or function of the larger protein to which it is fused. Protein tags are used for a variety of purposes, for example but not limited to, tags to facilitate purification, detection or visualization of the fusion proteins. Some peptide tags are removable by chemical agents or by enzymatic means, such as by target-specific proteolysis (e.g., by TEV protease, thrombin, Factor Xa or enteropeptidase) or intein splicing.

Affinity tags are appended to proteins to facilitate purification or visualization, and include chitin binding protein (CBP), maltose binding protein (MBP), and glutathione-S-transferase (GST), and the poly(His) tag. Solubilization tags are used to promote the proper folding of proteins, thereby improving solubility and minimizing protein precipitation. Solubilization tags include thioredoxin (TRX) and poly(NANP). Some affinity tags have dual roles as a solubilization agent, such as MBP and GST. Chromatography tags are used to improve the resolution of various separation techniques, such as polyanionic amino acid tags such as FLAG-tag. Epitope tags are short peptide sequences which are incorporated into a fusion protein because the availability of high-affinity antibodies to that peptide sequence. Epitope tags include V5-tag, Myc-tag, and HA-tag. These affinity tags have a variety of uses, including western blotting, immunofluorescence, immunoprecipitation and fusion protein purification. Some epitope tags also find use in the purification of antibodies that are specific for the epitope tag. Fluorescence tags are used to visual fusion protein production and protein subcellular localization, for example, under fluorescence microscopy. GFP and its many variants are commonly used fluorescence tags.

Depending on use, the terms “marker,” “reporter” and “tag” may overlap in definition, where the same protein or polypeptide can be used as either a marker, a reporter or a tag in different applications. In some scenarios, a polypeptide may simultaneously function as a reporter and/or a tag and/or a marker, all in the same recombinant gene or protein.

As used herein, the term “prokaryote” refers to organisms belonging to the Kingdom Monera (also termed Procarya), generally distinguishable from eukaryotes by their unicellular organization, asexual reproduction by budding or fission, the lack of a membrane-bound nucleus or other membrane-bound organelles, a circular chromosome, the presence of operons, the absence of introns, message capping and poly-A mRNA, a distinguishing ribosomal structure and other biochemical characteristics. Prokaryotes include subkingdoms Eubacteria (“true bacteria”) and Archaea (sometimes termed “archaebacteria”).

As used herein, the terms “bacteria” or “bacterial” refer to prokaryotic Eubacteria, and are distinguishable from Archaea, based on a number of well-defined morphological and biochemical criteria.

As used herein, the term “eukaryote” refers to organisms (typically multicellular organisms) belonging to the Kingdom Eucarya, generally distinguishable from prokaryotes by the presence of a membrane-bound nucleus and other membrane-bound organelles, linear genetic material (i.e., linear chromosomes), the absence of operons, the presence of introns, message capping and poly-A mRNA, a distinguishing ribosomal structure and other biochemical characteristics.

As used herein, the terms “mammal” or “mammalian” refer to a group of eukaryotic organisms that are endothermic amniotes distinguishable from reptiles and birds by the possession of hair, three middle ear bones, mammary glands in females, a brain neocortex, and most giving birth to live young. The largest group of mammals, the placentals (Eutheria), have a placenta which feeds the offspring during pregnancy. The placentals include the orders Rodentia (including mice and rats) and primates (including humans).

As used herein, the term “encode” refers broadly to any process whereby the information in a polymeric macromolecule is used to direct the production of a second molecule that is different from the first. The second molecule may have a chemical structure that is different from the chemical nature of the first molecule.

For example, in some aspects, the term “encode” describes the process of semi-conservative DNA replication, where one strand of a double-stranded DNA molecule is used as a template to encode a newly synthesized complementary sister strand by a DNA-dependent DNA polymerase. In other aspects, a DNA molecule can encode an RNA molecule (e.g., by the process of transcription that uses a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, the term “derived from” refers to a process whereby a first component (e.g., a first molecule), or information from that first component, is used to isolate, derive or make a different second component (e.g., a second molecule that is different from the first). For example, the mammalian codon-optimized Cas9 polynucleotides of the invention are derived from the wild type Cas9 protein amino acid sequence. Also, the variant mammalian codon-optimized Cas9 polynucleotides of the invention, including the Cas9 single mutant nickase and Cas9 double mutant null-nuclease, are derived from the polynucleotide encoding the wild type mammalian codon-optimized Cas9 protein.

As used herein, the expression “variant” refers to a first composition (e.g., a first molecule), that is related to a second composition (e.g., a second molecule, also termed a “parent” molecule). The variant molecule can be derived from, isolated from, based on or homologous to the parent molecule. For example, the mutant forms of mammalian codon-optimized Cas9 (hspCas9), including the Cas9 single mutant nickase and the Cas9 double mutant null-nuclease, are variants of the mammalian codon-optimized wild type Cas9 (hspCas9). The term variant can be used to describe either polynucleotides or polypeptides.

As applied to polynucleotides, a variant molecule can have entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also includes polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants includes nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

Variant polypeptides are also disclosed. As applied to proteins, a variant polypeptide can have entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also includes polypeptides that are portions or subsequences of the parent polypeptide, for example, unique subsequences (e.g., as determined by standard sequence comparison and alignment techniques) of the polypeptides disclosed herein are also encompassed by the invention.

In another aspect, polypeptide variants includes polypeptides that contain minor, trivial or inconsequential changes to the parent amino acid sequence. For example, minor, trivial or inconsequential changes include amino acid changes (including substitutions, deletions and insertions) that have little or no impact on the biological activity of the polypeptide, and yield functionally identical polypeptides, including additions of non-functional peptide sequence. In other aspects, the variant polypeptides of the invention change the biological activity of the parent molecule, for example, mutant variants of the Cas9 polypeptide that have modified or lost nuclease activity. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

As used herein, the term “conservative substitutions” in a nucleotide or amino acid sequence refers to changes in the nucleotide sequence that either (i) do not result in any corresponding change in the amino acid sequence due to the redundancy of the triplet codon code, or (ii) result in a substitution of the original parent amino acid with an amino acid having a chemically similar structure. Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the resulting polypeptide molecule.

The following are groupings of natural amino acids that contain similar chemical properties, where substitutions within a group is a “conservative” amino acid substitution. This grouping indicated below is not rigid, as these natural amino acids can be placed in different grouping when different functional properties are considered. Amino acids having nonpolar and/or aliphatic side chains include: glycine, alanine, valine, leucine, isoleucine and proline. Amino acids having polar, uncharged side chains include: serine, threonine, cysteine, methionine, asparagine and glutamine. Amino acids having aromatic side chains include: phenylalanine, tyrosine and tryptophan Amino acids having positively charged side chains include: lysine, arginine and histidine Amino acids having negatively charged side chains include: aspartate and glutamate.

As used herein, the terms “identical” or “percent identity” in the context of two or more nucleic acids or polypeptides refer to two or more sequences or subsequences that are the same (“identical”) or have a specified percentage of amino acid residues or nucleotides that are identical (“percent identity”) when compared and aligned for maximum correspondence with a second molecule, as measured using a sequence comparison algorithm (e.g., by a BLAST alignment, or any other algorithm known to persons of skill), or alternatively, by visual inspection.

The phrase “substantially identical,” in the context of two nucleic acids or polypeptides refers to two or more sequences or subsequences that have at least about 60%, about 80%, about 90%, about 90-95%, about 95%, about 98%, about 99% or more nucleotide or amino acid residue identity, when compared and aligned for maximum correspondence using a sequence comparison algorithm or by visual inspection. Such “substantially identical” sequences are typically considered to be “homologous,” without reference to actual ancestry. Preferably, the “substantial identity” between nucleotides exists over a region of the polynucleotide at least about 50 nucleotides in length, at least about 100 nucleotides in length, at least about 200 nucleotides in length, at least about 300 nucleotides in length, or at least about 500 nucleotides in length, most preferably over their entire length of the polynucleotide. Preferably, the “substantial identity” between polypeptides exists over a region of the polypeptide at least about 50 amino acid residues in length, more preferably over a region of at least about 100 amino acid residues, and most preferably, the sequences are substantially identical over their entire length.

The phrase “sequence similarity,” in the context of two polypeptides refers to the extent of relatedness between two or more sequences or subsequences. Such sequences will typically have some degree of amino acid sequence identity, and in addition, where there exists amino acid non-identity, there is some percentage of substitutions within groups of functionally related amino acids. For example, substitution (misalignment) of a serine with a threonine in a polypeptide is sequence similarity (but not identity).

As used herein, the term “homologous” refers to two or more amino acid sequences when they are derived, naturally or artificially, from a common ancestral protein or amino acid sequence. Similarly, nucleotide sequences are homologous when they are derived, naturally or artificially, from a common ancestral nucleic acid. Homology in proteins is generally inferred from amino acid sequence identity and sequence similarity between two or more proteins. The precise percentage of identity and/or similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are generally available.

As used herein, the terms “portion,” “subsequence,” “segment” or “fragment” or similar terms refer to any portion of a larger sequence (e.g., a nucleotide subsequence or an amino acid subsequence) that is smaller than the complete sequence from which it was derived. The minimum length of a subsequence is generally not limited, except that a minimum length may be useful in view of its intended function. The subsequence can be derived from any portion of the parent molecule. In some aspects, the portion or subsequence retains a critical feature or biological activity of the larger molecule, or corresponds to a particular functional domain of the parent molecule, for example, the DNA-binding domain, or the transcriptional activation domain. Portions of polynucleotides can be any length, for example, at least 5, 10, 15, 20, 25, 30, 40, 50, 75, 100, 150, 200, 300 or 500 or more nucleotides in length.

Polynucleotide subsequences of the invention have a variety of uses, for example but not limited to, as hybridization probes to identify polynucleotides of the invention, as PCR primers, or as donor sequences to be incorporated into a targeted homologous recombination event.

As used herein, the term “kit” is used in reference to a combination of articles that facilitate a process, method, assay, analysis or manipulation of a sample. Kits can contain written instructions describing how to use the kit (e.g., instructions describing the methods of the present invention), chemical reagents or enzymes required for the method, primers and probes, as well as any other components.

General Description

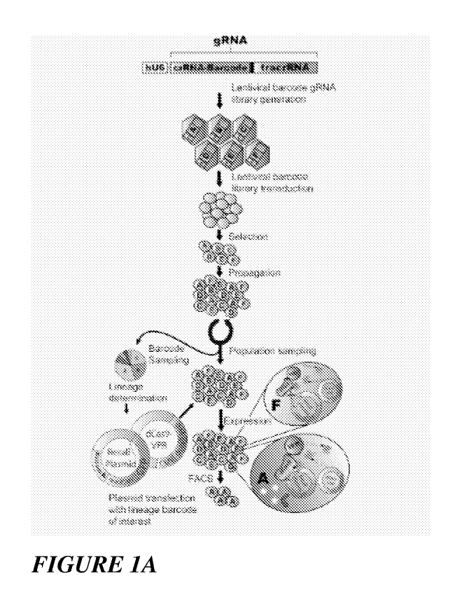

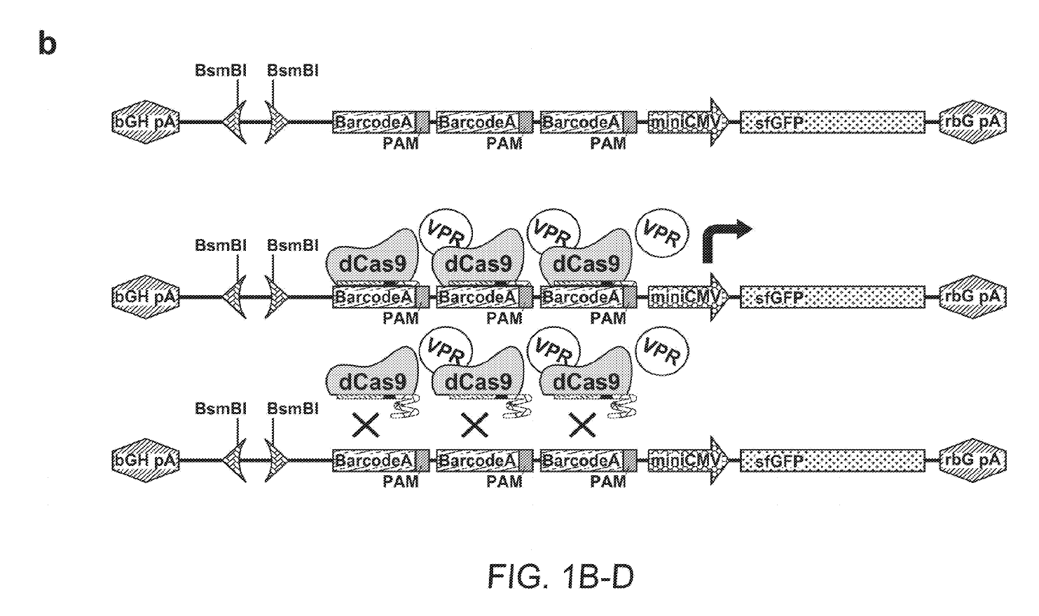

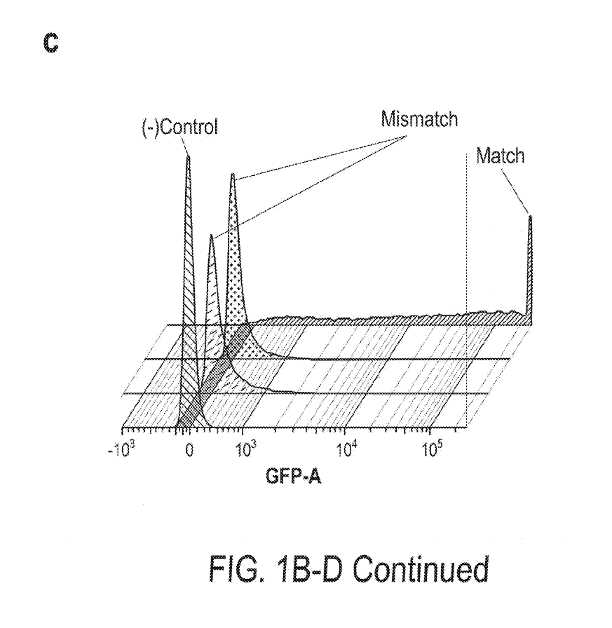

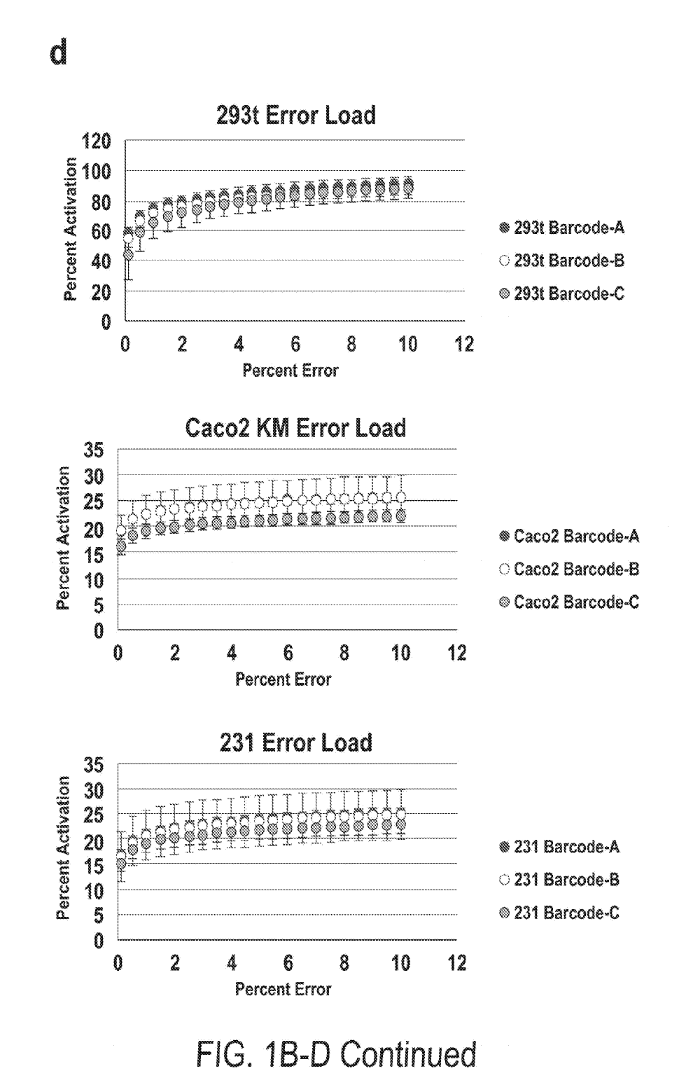

Disclosed herein are methods and compositions wherein each cell in a population is uniquely tagged with a stably integrated barcode-gRNA under control of a constitutive promoter. Following barcode instantiation, cells are permitted to proliferate and at intervals the genomically encoded barcode region is sequenced for quantitation of clonal barcodes; a parallel sample portion is archived for retroactive analysis. RNA sequencing of barcode gRNA can be performed directly in one example. Lineage dynamics may inform the identification of specific lineages of interest for subsequent gene activation in archival samples. Lineage-specific gene expression is accomplished by transfecting the entire population of cells with a plasmid containing a transcriptional activator variant of Cas9, dCas9-VPR, and a “Recall” plasmid encoding the lineage barcode of interest upstream of a gene to be activated. Only those cells containing the specified barcode-gRNA of interest, in coordination with dCas9-VPR, drive expression of the reporter gene. A schematic of the overall strategy of BAASE is shown in FIG. 1A.

There are many uses for this versatile tool, including driving lineage specific expression of a reporter, allowing lineage isolation via cell sorting. Other uses include driving lineage specific expression of a lethal protein, thereby allowing for targeted cell death of a specific lineage; use of an auxotrophic marker; use of a drug resistance gene/protein to allow for the targeted selection of a specific lineage of interest; or a differentiation marker to allow for lineage specific differentiation. Barcoded guide nucleotide can also be co-expressed with libraries of small non-coding RNA (microRNA) for functional assessment of microRNA.

Pooling libraries of miRNAs with barcoded gRNAs to track and allow for downnstream manipulation of these different cellular conditions. With the ability to derive lineages of interest from barcode clonal fitness analysis, recover whole cell populations from relevant time points, and isolate lineages from these time point samples, this allows for cellular and molecular analyses of pure lineage of interest populations. This ability to perform differential mutational analysis of a lineage among various time points and against other lineages gives unprecedented insight into evolutionary dynamics, bringing to light mutations, gene or protein expression changes, metabolic alterations and other molecular changes underlying specific clonal evolutionary trajectories.

Specifically, disclosed herein is a method of modulating expression of a gene of interest within a lineage of a select population of cells comprising: providing a population of cells; providing a vehicle, plasmid, vector or recombinant virus, or equivalent thereof, capable of stably expressing a guide nucleic acid, such as gRNA, comprising randomized barcodes, thereby producing a population of barcoded cells; allowing said barcoded cell to divide, thereby forming a barcoded progeny of cells; saving an aliquot of cells; identifying the barcode in a lineage of interest from the barcoded progeny of cells; reconstituting the aliquot of saved cells, and transforming the reconstituted aliquot of cells with a transcriptional element comprising a transcriptional effector, the barcode of the lineage of interest, and a gene of interest; utilizing the transcriptional effector to modify expression of the gene of interest within the lineage of interest.

Also disclosed herein is a platform for identifying a population of cells, the platform comprising: a population of cells; a vehicle, plasmid, vector or recombinant virus, or equivalent thereof, capable of stably expressing a guide nucleic acid, such as gRNA, comprising randomized barcodes; a transcriptional element comprising a transcriptional effector, the barcode of the lineage of interest, and a gene of interest.

DNA barcodes are sequences incorporated into cells and can be used to identify a specific cell into which the barcode was incorporated. Incorporating a distinct barcode for each cell allows for the pooling and parallel processing of the cells, which can later be separated based on their unique barcode. Every barcode in a set is unique, that is, any two barcodes chosen out of a given set differ in at least one nucleotide position.

Barcoded cells can be constructed, for example, using DNA constructs. Examples of barcoding cells are known in the art, and can be found, for example, published PCT Application WO2013033721, herein incorporated by reference in its entirety. Also disclosed is US Patent Application US20160020085, also incorporated by reference in its entirety for its disclosure concerning barcodes.

Various sets of barcodes have been reported in the literature. Several researchers have used sets that satisfy the conditions imposed by a Hamming Code [Hamming, R. W., Bell System Technical Journal v. XXIX no. 2, pp. 147-160, April 1950, Hamady et. al. (2008), Nature Methods v. 5 no. 3, pp 235-237, Lefrancois et. al. (2009), BMC Genomics v. 10 no. 37 pp 1-18]. Others have used sets that satisfy more complex conditions than a Hamming Code but share the similar guarantee of a certain minimal pairwise Hamming distance [Fierer et. al. (2008), PNAS v. 105 no. 46 pp 17994-17999, Krishnan et al. (2011), Electronics Letters v. 47 no. 4 pp. 236-237]. Such barcodes are not useful with a sequence that has an insertion or deletion in the region including the barcode. As an alternative to Hamming-distance based barcodes, others have selected sets of barcodes which satisfy a minimum pairwise edit distance. Sets of such barcodes can work with insertion, deletion or substitution errors in the read of a barcode sequence.

Various modified nucleotide-guided protein systems which are used to modulate gene expression can be used with the methods disclosed herein, as well as their modified variants. These systems are known to those of skill in the art. Examples include those found in the following references, which are herein incorporated by references for their teaching concerning nucleotide-guided protein systems: Bibikova, M, Golic, M, Golic, K G and Carroll, D (2002). Targeted chromosomal cleavage and mutagenesis in Drosophila using zinc-finger nucleases. Genetics 161: 1169-1175; Zetsche, B, Gootenberg, J S, Abudayyeh, O O, Slaymaker, I M, Makarova, K S, Essletzbichler, P et al. (2015). Cpf1 Is a Single RNA-Guided Endonuclease of a Class 2 CRISPR-Cas System. Cell 163: 759-771; Moscou, M J and Bogdanove, A J (2009). A simple cipher governs DNA recognition by TAL effectors. Science 326: 1501; Boch, J, Scholze, H, Schornack, S, Landgraf, A, Hahn, S, Kay, S et al. (2009). Breaking the code of DNA binding specificity of TAL-type III effectors. Science 326: 1509-1512; Shmakov, S, Abudayyeh, 00, Makarova, K S, Wolf, Y I, Gootenberg, J S, Semenova, E et al. (2015). Discovery and Functional Characterization of Diverse Class 2 CRISPR-Cas Systems. Mol Cell 60: 385-397; Mali, P, Aach, J, Stranges, P B, Esvelt, K M, Moosburner, M, Kosuri, S et al. (2013). CAS9 transcriptional activators for target specificity screening and paired nickases for cooperative genome engineering. Nat Biotechnol 31: 833-838; Cong, L, Ran, F A, Cox, D, Lin, S, Barretto, R, Habib, N et al. (2013). Multiplex genome engineering using CRISPR/Cas systems. Science 339: 819-823.

A specific example of a nucleotide guided protein system is the CRISPR system. The CRISPR/Cas or the CRISPR-Cas system (both terms are used interchangeably throughout this application) can be used to identify and/or separate a group or a lineage of cells based on the unique barcode incorporated into the population of cells or parent(s) of the population of cells. For example, when cell passaging is carried out, lineage from a specific parent cell into which a unique barcode was incorporated can be identified and isolated. The CRISPR/Cas system can comprise a guide nucleic acid, such as a guide RNA, or single guide RNA (referred to herein as gRNA or sgRNA). The gRNA can comprise a crRNA and a tracrRNA segment under the control of a promoter, for example. As disclosed herein, the crRNA segment can comprise the randomized barcode. The crRNA segment can be upstream of a tracrRNA, and can be under the control of a promoter. gRNAs each carrying a unique barcode can be introduced into a population of cells. Those cells can later be isolated based on their barcode.

A single Cas enzyme can then be used which recognizes a barcode of interest. For example, if a given population of cells is of particular interest, one can determine the unique barcode found in that population of cells, then utilize Cas to select those cells from an saved aliquot of cells. In other words, the Cas enzyme can be recruited to a specific DNA target, such as the barcode, using the gRNA molecule. Disclosed is published PCT application WO2015/089486A2, which discusses the CRISPR/Cas system, and is herein incorporated by reference in its entirety.

Using the methods and platforms disclosed herein, any population of cells which have been barcoded can later be identified. For example, an aliquot of cells can be saved at any time point during cell division. The cells can be saved before dividing, after dividing, or both before and after division. Alternatively, the cells don't need to be divided at all, and the aliquot can be saved at any time point during experimentation with the cells.

The Cas system can comprise a transcriptional element, which allows for the identification of a population of cells comprising the desired barcode. The transcriptional element can be in the form of a plasmid, for example. The transcriptional element can comprise a transcriptional effector, the barcode of the lineage of interest, and a gene of interest, as well as any regulatory sequences necessary for transcriptional regulation via nucleotide dependent sequence specific DNA binding protein, such as a PAM site for Cas9. One of skill in the art will understand how to obtain and use such regulatory sequences. In one example, the barcode of the lineage of interest can be upstream the gene of interest, and can further comprise a regulatory sequence as well. The transcriptional effector can be used to modulate expression of the gene of interest, such that the population of cells can be readily identified and/or modulated based on the gene of interest. The barcode in the transcriptional element is used by the Cas system to form a match with those cells which comprise an identical barcoded segment from the gRNA. The transcriptional effector can be any nucleic acid capable of modulating expression of the gene of interest. The transcriptional effector can comprise a cleavage domain (catalyzing cleavage with or without a frameshift), an epigenetic modification domain, a transcriptional activation domain, or a transcriptional repressor domain.

As used herein, a “cleavage domain” refers to a domain that cleaves DNA. The cleavage domain can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., 51 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) can be used as a source of cleavage domains. In one example, the cleavage domain can be derived from a type II-S endonuclease. Type II-S endonucleases cleave DNA at sites that are typically several base pairs away the recognition site and, as such, have separable recognition and cleavage domains. These enzymes generally are monomers that transiently associate to form dimers to cleave each strand of DNA at staggered locations. Non-limiting examples of suitable type II-S endonucleases include Bfil, Bpml, Bsal, Bsgl, BsmBI, Bsml, BspMI, Fokl, Mboll, and Sapl.

The transcriptional effector domain of the transcriptional element can be an epigenetic modification domain. In general, epigenetic modification domains alter histone structure and/or chromosomal structure without altering the DNA sequence. Changes in histone and/or chromatin structure can lead to changes in gene expression. Examples of epigenetic modification include, without limit, acetylation or methylation of lysine residues in histone proteins, and methylation of cytosine residues in DNA. Non-limiting examples of suitable epigenetic modification domains include histone acetyltansferase domains, histone deacetylase domains, histone methyltransferase domains, histone demethylase domains, DNA methyltransferase domains, and DNA demethylase domains.

In embodiments in which the effector domain is a histone acetyltansferase (HAT) domain, the HAT domain can be derived from EP300 (i.e., binding protein p300), CREBBP (i.e., CREB-binding protein), CDY1, CDY2, CDYL1, CLOCK, ELP3, ESA1, GCN5 (KAT2A), NATI, KAT2B, KAT5, MYST1, MYST2, MYST3, MYST4, NCOA1, NCOA2, NCOA3, NCOAT, P/CAF, Tip60, TAFI1250, or TF3C4.

In embodiments wherein the effector domain is an epigenetic modification domain and the CRISPR/Cas-like protein is derived from a Cas9 protein, the Cas9-derived can be modified such that its endonuclease activity is eliminated. For example, the Cas9-derived can be modified by mutating the RuvC and HNH domains such that they no longer possess nuclease activity.

The effector domain of the fusion protein can be a transcriptional activation domain. In general, a transcriptional activation domain interacts with transcriptional control elements and/or transcriptional regulatory proteins (i.e., transcription factors, RNA polymerases, etc.) to increase and/or activate transcription of a gene. In some embodiments, the transcriptional activation domain can be, without limit, dCas9-VPR, a herpes simplex virus VP16 activation domain, VP64 (which is a tetrameric derivative of VP16), a NFKB p65 activation domain, p53 activation domains 1 and 2, a CREB (cAMP response element binding protein) activation domain, an activation domain, and an NFAT (nuclear factor of activated T-cells) activation domain. Other nucleotide-guided proteins include cpf1 and NgAgo.

The transcriptional activation domain can be 0a14, Gcn4, MLL, Rtg3, 01n3, Oaf1, Pip2, Pdr1, Pdr3, Pho4, and Leu3. The transcriptional activation domain may be wild type, or it may be a modified version of the original transcriptional activation domain. In some embodiments, the effector domain of the fusion protein is a dCas9-VPR transcriptional activation domain. The Cas9-derived protein can be modified such that its endonuclease activity is eliminated. For example, the Cas9-derived can be modified by mutating the RuvC and HNH domains such that they no longer possess nuclease activity.

The effector domain of the fusion protein can be a transcriptional repressor domain. In general, a transcriptional repressor domain interacts with transcriptional control elements and/or transcriptional regulatory proteins (i.e., transcription factors, RNA polymerases, etc.) to decrease and/or terminate transcription of a gene. Non-limiting examples of suitable transcriptional repressor domains include inducible cAMP early repressor (ICER) domains, Kruppel-associated box A (KRAB-A) repressor domains, YY1 glycine rich repressor domains, Sp1-like repressors, E(spl) repressors, IKB repressor, and MeCP2. In embodiments wherein the effector domain is a transcriptional repressor domain and the CRISP R/Cas-like protein is derived from a Cas9 protein, the Cas9-derived protein can be modified as discussed herein such that its endonuclease activity is eliminated. For example, the cas9 can be modified by mutating the RuvC and HNH domains such that they no longer possess nuclease activity.

The fusion protein can further comprise at least one additional domain Non-limiting examples of suitable additional domains include nuclear localization signals, cell-penetrating or translocation domains, and marker domains.

The gene of interest within the transcriptional element can be a marker, such as a reporter. A variety of marker types are commonly used, and can be for example, visual markers such as color development, e.g., lacZ complementation (β-galactosidase) or fluorescence, e.g., such as expression of green fluorescent protein (GFP) or GFP fusion proteins, RFP, BFP, selectable markers, phenotypic markers (growth rate, cell morphology, colony color or colony morphology, temperature sensitivity), auxotrophic markers (growth requirements), antibiotic sensitivities and resistances, molecular markers such as biomolecules that are distinguishable by antigenic sensitivity (e.g., blood group antigens and histocompatibility markers), cell surface markers (for example H2KK), enzymatic markers, and nucleic acid markers, for example, restriction fragment length polymorphisms (RFLP), single nucleotide polymorphism (SNP) and various other amplifiable genetic polymorphisms.

Cells in the lineage of interest can be selected in a variety of ways, known to those of skill in the art. For example, cells can be selected on the basis of phenotype, wherein the phenotype can be created from the gene of interest. Selecting the cells on the basis of phenotype can comprise selecting the cells on the basis of protein expression, RNA expression, or protein activity. In some cases selecting the cells on the basis of the phenotype comprises fluorescence activated cell sorting, affinity purification of cells, or selection based on cell motility. For example, cell sorting can be done using single cell sorting, fluorescent activated cell sorting (FACS), physical cell manipulation, laser capture, or magnetic cell sorting.

In one example, prior to identifying the barcode in a lineage of interest, the cells are exposed to a candidate agent. Candidate agents can be tested to determine their activity in a cell. The terms “candidate agent” or “drug” as used herein encompass small molecules (e g, small organic molecules), peptides, carbohydrates, antibodies or antibody fragments, or nucleic acid sequences, including DNA and RNA sequences. In one example, the candidate agent can be monitored to determine how it interacts with a target molecule produced by the cell of interest. “Target molecule” as used herein, encompasses peptides, proteins and nucleic acid sequences, both DNA and RNA, produced by, or present in mammalian cells, bacteria or viruses. Target molecules suitable for use in the present invention typically possess a biological activity, or function, which is critical for the growth, proliferation or differentiation of a eukaryotic cell, or of a bacteria or virus capable of entering and infecting a eukaryotic cell. Such target molecules include, for example, proteins necessary for viral replication or viral gene expression, eukaryotic transcription factors, enzymes such as protein kinases, and cytokines involved in cellular differentiation.

Specifically, disclosed herein is a method of generating a population of cells that display a desired characteristic when exposed to a candidate agent, the method comprising: providing a population of cells; providing a vehicle, plasmid, vector or recombinant virus, or equivalent thereof, capable of stably expressing a guide nucleic acid comprising randomized barcodes, thereby producing a population of barcoded cells; saving an aliquot of cells; exposing the barcoded cells to one or more candidate agents; identifying a desired characteristic in a barcoded cell exposed to a candidate agent; reconstituting the aliquot of cells and exposing the reconstituted aliquot of cells to a nucleic acid comprising a transcriptional activator, a barcode, and a gene of interest, wherein the barcode is the same as that of the barcoded cell with the desired characteristic; utilizing the transcriptional activator to drive expression of the gene of interest; identifying and selecting barcoded cells with the desired characteristic; and allowing the selected barcoded cell to divide, thereby forming generating a population of cells that display a desired characteristic when exposed to a candidate agent.

The candidate agent can cause modulation in the activity of a cell or in a target molecule of the cell. For example, the candidate agent can upregulate, downregulate, cause apoptosis, or cell multiplication. Once a cell, or population of cells, has been identified as being of interest based on its interaction with a candidate agent, that cell or population of cells can be sequenced to determine its unique barcode.

Many types of screens and selection mechanisms can also be used with the methods and platforms disclosed herein. Screens for resistance to viral or bacterial pathogens may be used to identify genes that prevent infection or pathogen replication. These screens can also be used to identify epigenetic changes. As in drug resistance screens, survival after pathogen exposure provides strong selection. In cancer, negative selection screens may identify “oncogene addictions” in specific cancer subtypes that can provide the foundation for molecular targeted therapies. For developmental studies, screening in human and mouse pluripotent cells may pinpoint genes required for pluripotency or for differentiation into distinct cell types. To distinguish cell types, fluorescent or cell surface marker reporters of gene expression may be used and cells may be sorted into groups based on expression level. Gene-based reporters of physiological states, such as activity-dependent transcription during repetitive neural firing or from antigen-based immune cell activation, may also be used. Any phenotype that is compatible with rapid sorting or separation may be harnessed for pooled screening. Screening may also be used as a diagnostic tool: Screens can be used to identify cell lineages with sensitivity or resistance to specific therapeutic agents. With patient-derived iPS cells, genome-wide libraries may be used to examine multi-gene interactions (similar to synthetic lethal screens) or how different loss-of-functions mutations accumulated through aging or disease can interact with particular drug treatments.

Disclosed herein are methods of determining a chemotherapy resistant cell, the method comprising the steps of: a) obtaining tumor cells from a patient undergoing chemotherapy; b) labeling the tumor cells with a library of expressed barcodes; c) culturing the tumor cells of step b); d) treating the cells with the same chemotherapy treatment as the patient; e) monitoring growth dynamics of the tumor cells; f) determining a chemotherapy resistant cell. Also disclosed are methods of determining patient treatment regimes based on the results. For example, a patient who is found to have drug resistance to a certain chemotherapy agent can be treated differently based on the results thereof.

The tumor cells can be derived from multiple methods known to those of skill in the art. For example, the tumor cells can be are derived from the patient and cultured ex vivo. Each of the expressed barcodes of step b) are unique. Monitoring growth dynamics can comprise determining those cells that survive the chemotherapy treatment of step d). It can also comprise determining those cells that survive longer than other cells when given the chemotherapy treatment of step d).

The chemotherapy resistant cell can be isolated and subjected to various studies to determine its resistant level, what it is resistant to, and what other treatment options might be useful (i.e., what the cell isn't resistant to).

Methods of identifying and characterizing new and useful drug candidates include the isolation of natural products or synthetic preparation, followed by testing against either known or unknown targets. These techniques are known to those of skill in the art. See for example WO 94/24314, Gallop et al., J. Med. Chem. 37(9):1233 (1994); Gallop et al., J. Med. Chem. 37(10):1385 (1994); Ellman, Acc. Chem. Res. 29:132 (1996); Gordon et al., E. J. Med. Chem. 30:388s (1994); Gordon et al., Acc. Chem. Res. 29:144 (1996); WO 95/12608, all of which are incorporated by reference.

Disclosed herein is a kit for use in identifying a population of cells, the kit comprising: a population of cells a vehicle, plasmid, vector or recombinant virus, or equivalent thereof, capable of stably expressing a guide nucleic acid comprising randomized barcodes; and a nucleic acid comprising a transcriptional activator, the barcode of the lineage of interest, and a gene of interest. The kit disclosed herein can comprise any one or more of the elements disclosed in the above methods and platforms.

In some embodiments, the kit comprises a plasmid system and instructions for using the kit. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

The examples below are intended to further illustrate certain aspects of the methods and compounds described herein, and are not intended to limit the scope of the claims.

### Examples

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Baase

BAASE is a method that can enable identification and collection, as well as modulation, of cells of a particular lineage (derived from a common ancestor cell), alongside lineage-specific expression of a gene of interest (See FIG. 1). The method brings together DNA-barcoding and CRISPR/Cas9 technologies. This method consists of: (i) a barcoded population of cells with a DNA construct composed of a randomized barcoded crRNA segment upstream of a tracrRNA under control of a promoter; (ii) over a time course, a portion of the barcoded sample is processed for relative clonal barcode frequency; (iii) concurrent with (ii), an aliquot of the sample is saved as a freezer stock; (iv) upon clonal analysis from (ii), a lineage of interest can be derived. Samples from (iii) can be reconstituted and the whole population can be transformed/transfected with a plasmid containing a transcriptional activator variant of dCas9 (such as dCas9-VPR) and the lineage barcode of interest upstream of a gene of interest. Only those cells containing the barcode-gRNA of interest, in coordination with transcriptional activator dCas9, will bind to the barcode of interest contained on the plasmid and drive expression of a gene of interest. This system allows for longitudinal clonal analysis, reconstitution of previous time point populations, and lineage specific expression of a gene of interest. One current utility for this versatile method revolves around driving lineage specific expression of a reporter, allowing lineage isolation via cell sorting. Deriving lineages of interest from clonal fitness analysis, recovery of whole cell populations from relevant time points, and lineage isolation from these time point samples will allow for unprecedented lineage purity for downstream molecular and cellular analyses.

### Example 2: Control of Lineage-Specific Gene Expression by Functionalized gRNA Barcodes

To demonstrate lineage-specific expression of a fluorescent reporter by BAASE, three independent populations expressing a single known barcode gRNA (Bg), Bg-A, Bg-B, Bg-C, were expressed. Cells were transduced at a multiplicity of infection (MOI) of 0.1 to minimize instances of integration of more than one barcode. Cells containing stably integrated barcode sequences were selected by BFP⁺ expression (FIG. 1a). Three different Recall plasmids (Recall A-C) each containing one of the three corresponding barcode regions and PAM site upstream of a miniCMV promoter and sfGFP (FIG. 1b) were then expressed. These barcode populations were then transfected with each of the Recall plasmids+dCas9-VPR plasmid independently, causing instances of either match or mismatch with regards to the barcoded gRNA and Recall plasmid (FIG. 1a). After 48 hours, GFP expression was assessed via flow cytometry. The results showed that barcoded cell populations transfected with a matching Recall plasmid were able to activate expression of the fluorescent reporter, while only nominal expression was present in the instances of mismatch (FIG. 1c). This robust and easy-to-use platform can be deployed in a variety of cell types. To assess the efficiency of lineage-specific GFP activation in the match population and compare with non-specific activation of mismatch population, the error load associated with deploying the system in HEK293T, Caco2, and MB-MDA-231 cells was quantified. 80% of the lineage-specific GFP cells could be identified in HEK293T with 2% false positive activation. Error rates also remained low in Caco2, and MB-MDA-231 cells, although GFP activation was significantly lower due to less efficient plasmid transfection in these cell types (FIG. 1d).

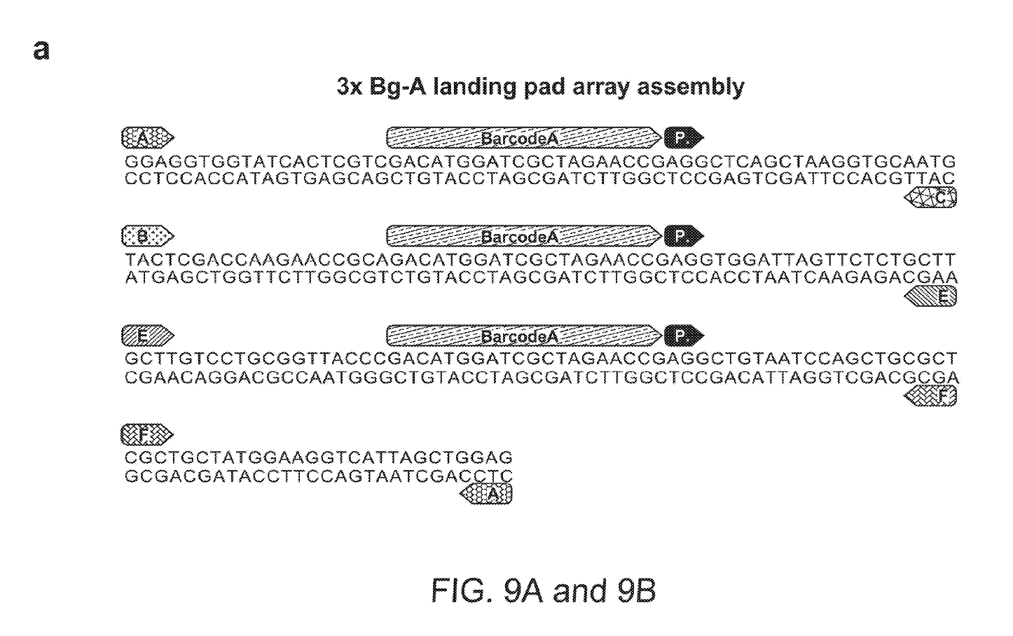

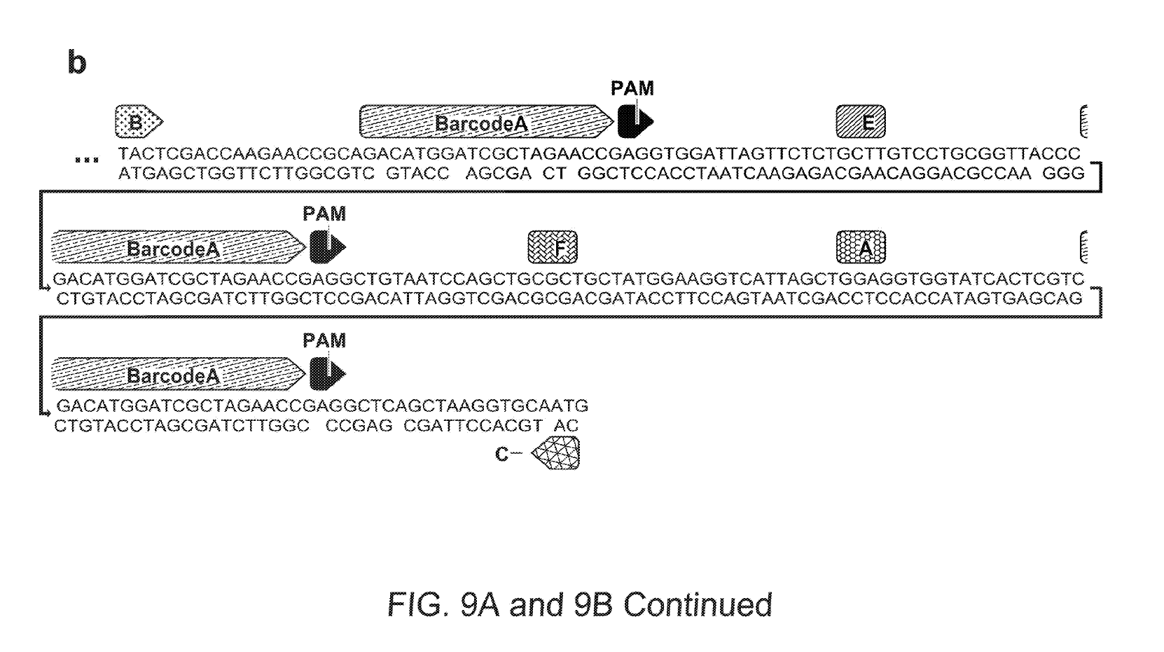

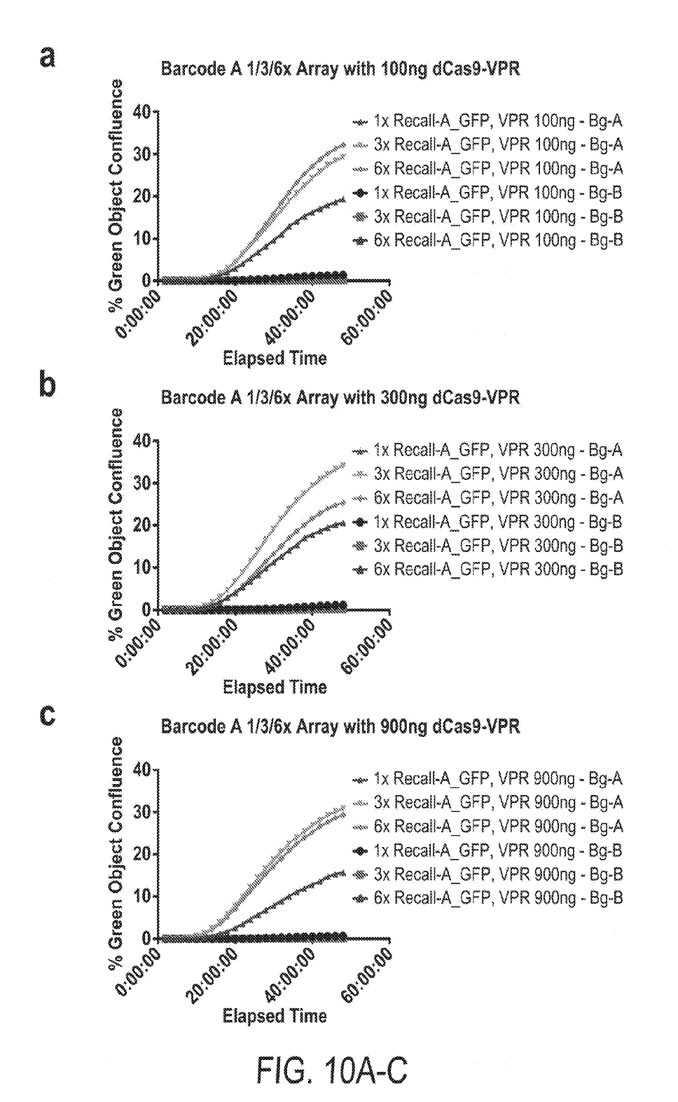

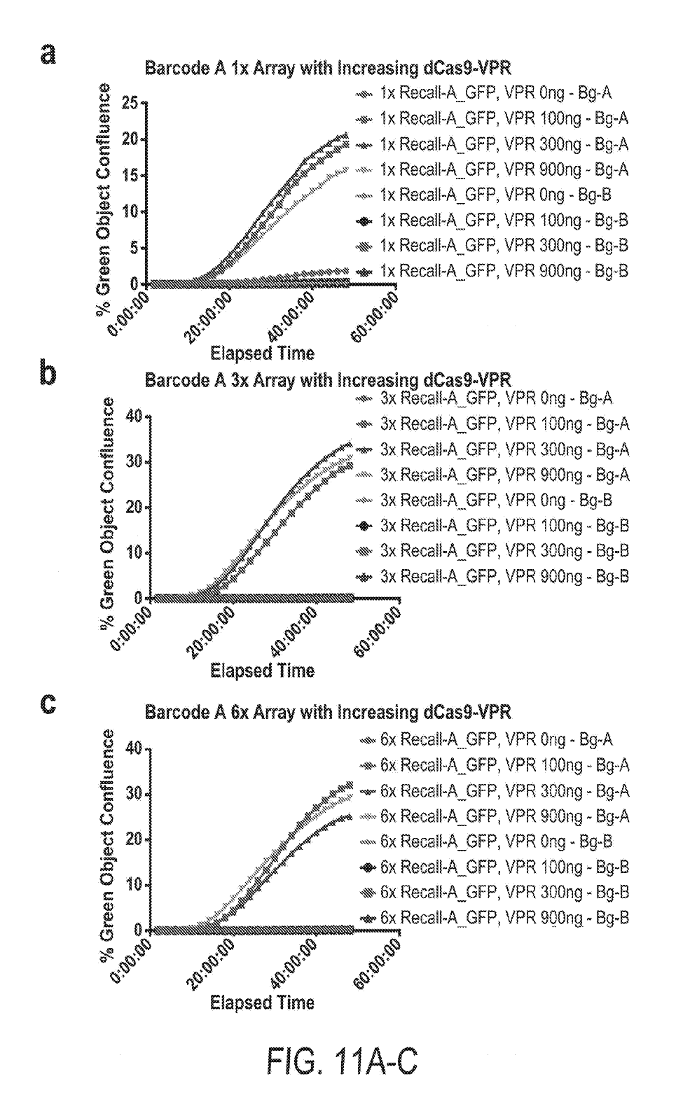

To optimize lineage-specific activation with the Recall plasmid, alternative designs were tested with varying numbers of barcode recognition sites (lx, 3×and 6×). In addition, both Recall and dCas9 VPR plasmid were titrated to determine optimal amounts to activate barcode-driven expression (FIGS. 9-11).

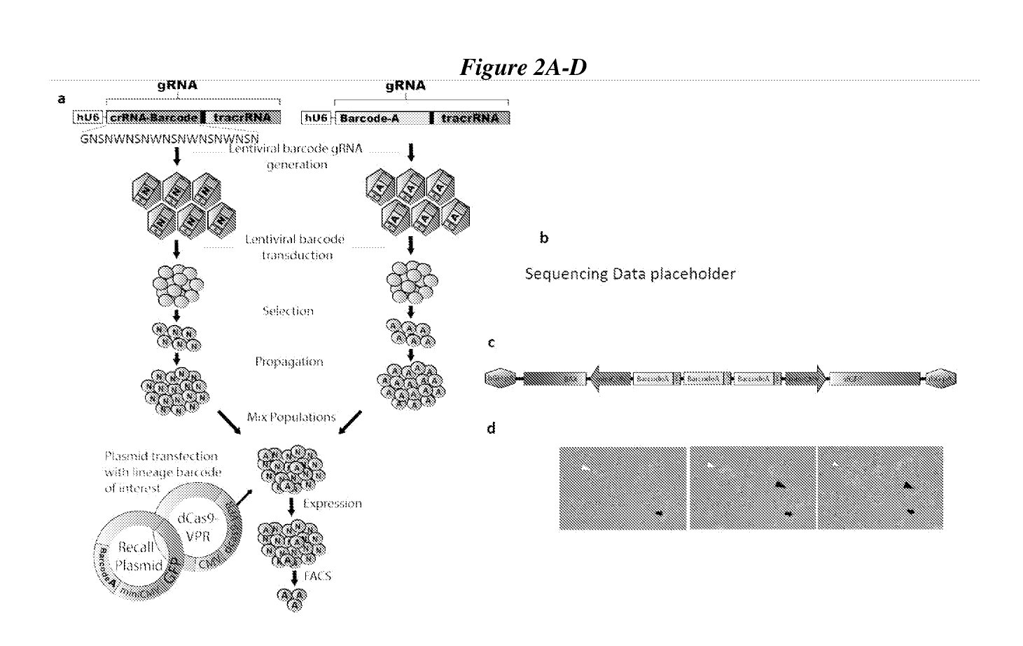

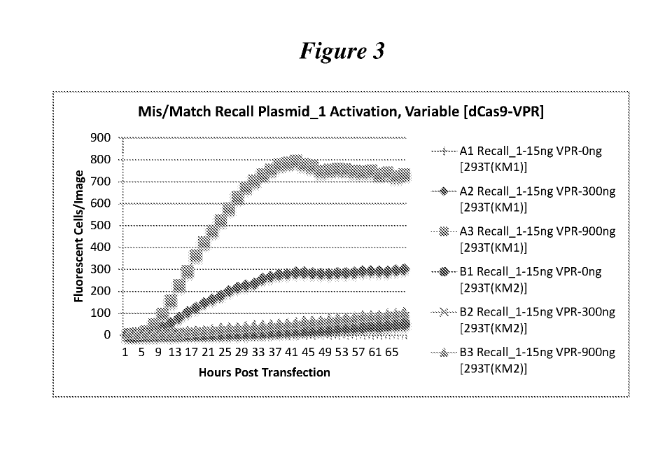

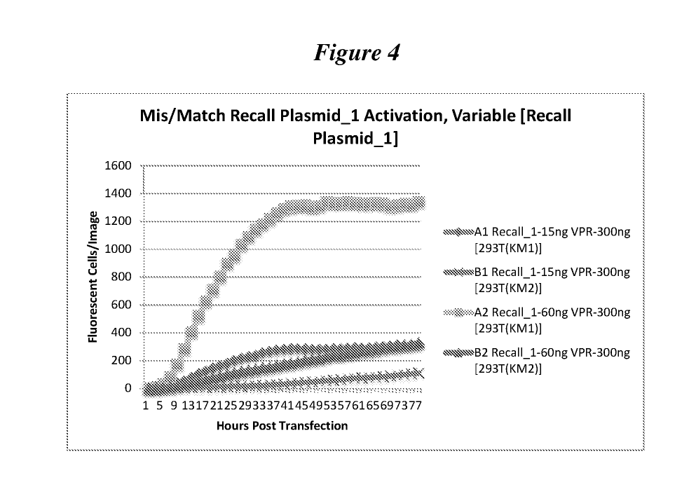

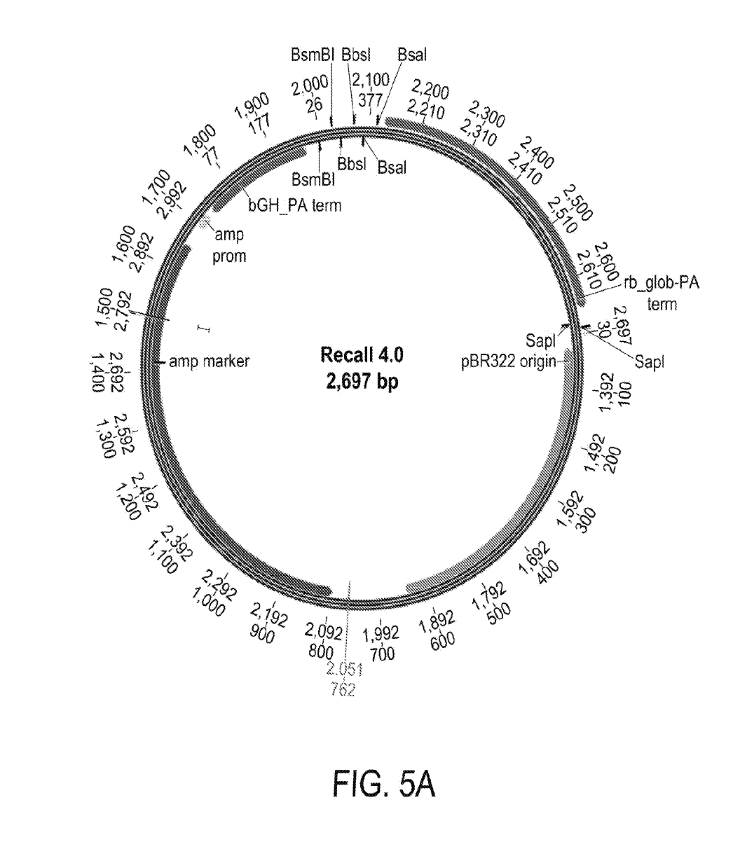

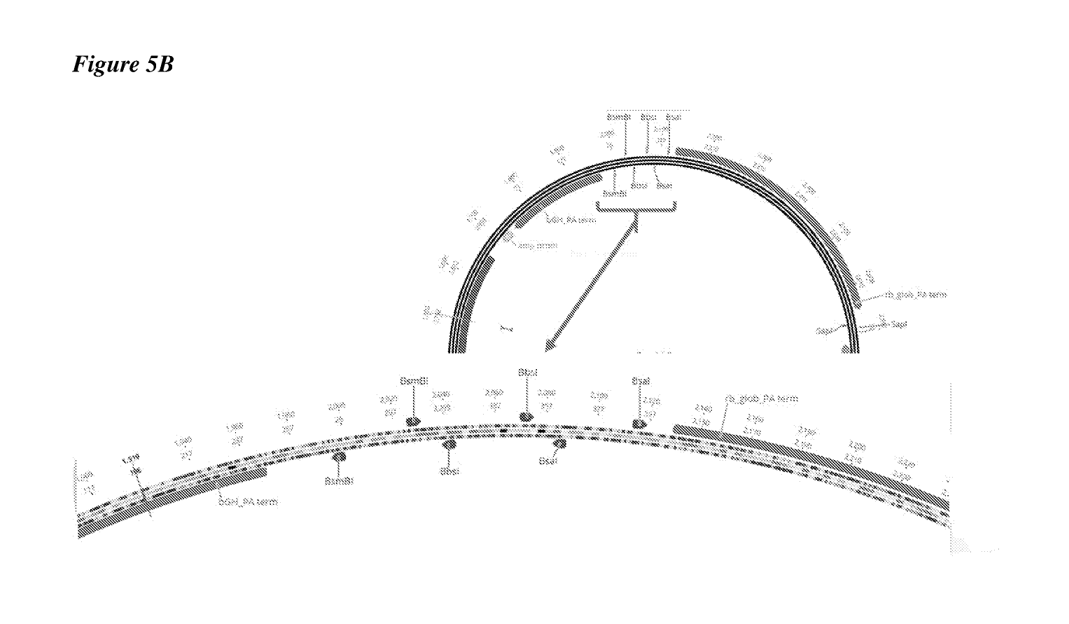

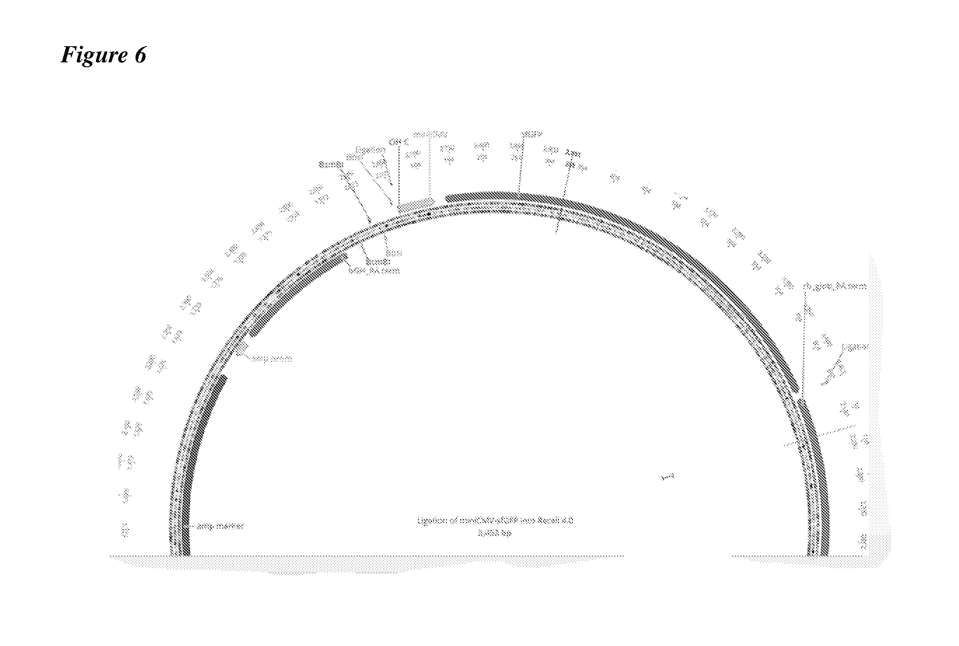

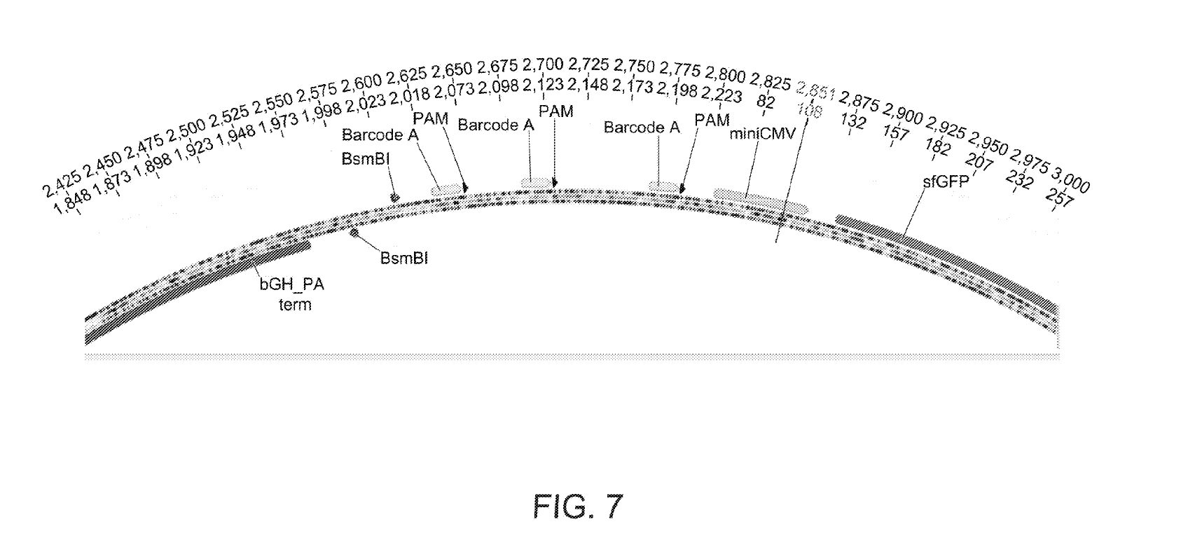

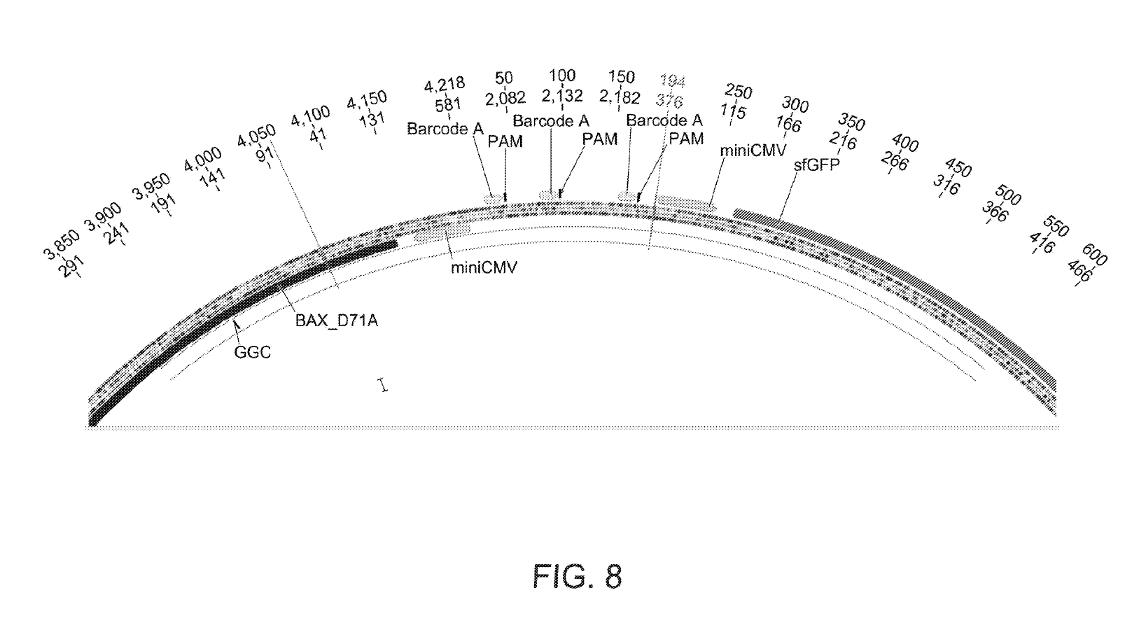

To confirm the specificity and efficiency of lineage-specific expression, recall was tested in the presence of a large diverse barcoded population. A high-diversity barcode gRNA library was constructed with the template: GNSNWNSNWNSNWNSNWNSN (SEQ ID NO: 1), having a diversity potential greater than 500,000,000 unique sequences (FIG. 2). This gRNA library was ligated into a gRNA expression lentiviral transfer vector and assembled into a pooled gRNA barcoded lentivirus. Following transduction, stably integrated BFP⁺ cells were collected, yielding a high diversity population of <10⁶ barcoded cells. Cells from the Bg-A population were then spiked into the high diversity library at 1/100 and 1/1000 dilution and grown overnight. The spiked populations were then co-transfected with the Recall and dCas9-VPR plasmids and sorted via FACS for GFP expression. Sorted cells were subcultured and genomic DNA was isolated for sequencing. To ensure quantitative assessment of barcodes, templates were (i) extended with primers containing unique molecular indices, (ii) reverse extended with a biotinylated primer, (iii) streptavidin purified, and (vi) thermocycled with primers containing Illumina adaptor sequences. Barcode sequencing of the population confirms that BAAR identified the fraction of cells carrying the reference Bg-A barcode from within the high diversity population (FIG. 2b-c).

Beyond the control of fluorescent reporter gene expression, this system can be functionalized to express any set of genes in a lineage-specific manner. To explore the multifunctionality of this system we sought to perturb the cell fates of specific lineages, by driving lineage-specific expression of the pro-apoptotic protein, Bax (FIG. 2d). Time lapse fluorescent imaging reveals lineage-specific gene expression of GFP and subsequent apoptosis of fluorescing cells (FIG. 2d). Co-staining for annexin confirms activation of apoptotic signaling (FIG. 2d).

The demonstration that expressed gRNA barcodes can be used to efficiently perform lineage-specific manipulation of gene expression opens up the possibility for a broad range of studies investigating the potential of lineage-specific perturbations within the context of a heterogeneous, evolving cell population. The ability to concurrently track clonal fitness dynamics and generate lineage-specific genomic and transcriptomic data over longitudinal studies will provide unprecedented insight into cancer adaptation and other diseases with an evolutionary basis.

High-Complexity Barcode-gRNA Library Construction.

The following 60 base-pair oligonucleotide containing a 19 nucleotide semi-random sequence corresponding the barcode guide-RNA and reverse extension primer was ordered from Integrated DNA Technologies.

GAGCCTGAAGACCTCACCGNSNWNSNWNSNWNSNWNSNGTTTTAGCGTCTT CCATGCGCA (SEQ ID NO: 2), TGCGCATGGAAGACGCTAAAAC (SEQ ID NO: 12). An extension reaction was performed to generate the double stranded barcode-gRNA oligo. The double stranded product contains two BbsI sites that, after digestion, generate complimentary overhangs for ligation into the gRNA expression transfer vector pKLV-U6gRNA(BbsI)-PGKpuro2ABFP (Addgene). 1 μg of BbsI digested gRNA expression transfer vector was ligated with digested barcode-gRNA insert in a molar ratio 1:7. This reaction was cleaned and concentrated in 6 μl using the Zymo DNA Clean & Concentrator™ kit and transformed into electrocompetent SURE 2 cels (Agilent). Transformants were inoculated into 500 ml of 2×YT containing 100 μg/ml carbenicillin for outgrowth overnight at 37° C. Transformation efficiency was calculated via dilution plating and shown to be approximately 7e8 cfu/μg.

Mock Barcode-gRNA Construction.

Three different discrete known barcode-gRNA lentiviral expression vectors were generated with the sequences: A) GACATGGATCGCTAGAACCG (SEQ ID NO: 3), B) GTCAAGGTAGCTAAGTAGCG (SEQ ID NO: 4), C) GTCAAGCGTGCAATGGTAGC (SEQ ID NO: 5). To accomplish this, oligo pairs with complimentary barcode sequences and the appropriate overhang sequences were mixed and cloned into the BbsI digested pKLV-U6gRNA(BbsI)-PGKpuro2ABFP transfer vector at a 10:1 molar ratio:

A)
(SEQ ID NO: 6)
CACCGACATGGATCGCTAGAACCGGT,
(SEQ ID NO: 7)
TAAAACCGGTTCTAGCGATCCATGTC,
B)
(SEQ ID NO: 8)
CACCGTCAAGGTAGCTAAGTAGCGGT,
(SEQ ID NO: 9)
TAAAACCGCTACTTAGCTACCTTGAC,
C)
(SEQ ID NO: 10)
CACCGTCAAGCGTGCAATGGTAGCGT,
(SEQ ID NO: 11)
TAAAACGCTACCATTGCACGCTTGAC.

Lentiviral Assembly Lentiviral assembly was accomplished using the GeneCopeia Lenti-Pac™ HIV Expression Packaging Kit (cat# HPK-LvTR-20). Two days prior to lentiviral transfection HEK293T cells were plated onto a 10 cm dish at 1.5 million cells and cultured in 10 ml DMEM supplemented with 10% heat inactivated FBS. 48 hours after plating, cells were 70-80% confluent and transfected with 15 μl of EndoFectin and a mix of 2.5 μg of pKLV-U6Barcode-gRNA-PGKpuro2ABFP and 2.5 μg of Lenti-Pac™ HIV mix (GeneCopoeia). The media was replaced 14 hours post transfection with 10 ml DMEM supplemented with 5% heat inactivated FBS and 20 μl TiterBoost™(GeneCopoeia) reagent. Media containing viral particles was collected at 48 and 72 hours post transfection, centrifuged at 500 g for 5 minutes, and filtered through a 45 μm polyethersulfone (PES) low protein-binding filter. Filtered supernatant was aliquoted and stored at −80° C. for later use.

Barcoding Cell Lines.

Cell lines HEK 293T, MB-MDA-231, and Caco-2 cell lines were cultured in DMEM medium supplemented with 10% FBS and 1% penicillin-streptomycin. Cells were transduced with the pKLV-U6Barcode-gRNA-PGKpuro2ABFP lentivirus using 1 μg/ml polybrene. After 48 h incubation, BFP⁺ cells were isolated by FACS. To reduce the likelihood that two viral particles enter a single cell, the lentiviral transduction multiplicity of infection was kept below 0.1.

Barcode Amplification.

After lineage isolation, cell populations of interest were harvested and genomic DNA was extracted using the PureLink® Genomic DNA Mini Kit (Thermo Fisher cat# K1820-01). Barcode sequences were amplified using PCR and sent for NGS. Primer sequences contained both flanking barcode annealing regions and Illumina adaptor/index sequence. For each PCR reaction, 250 ng of genomic DNA was used as a template.

Recall Plasmid Assembly.

The Recall plasmid was constructed by using standard restriction cloning to combine a gBlock® containing three tandem type IIS restriction sites (BsmBI, BbsI, BsaI) flanked by terminators with an amplicon containing a bacterial replication origin and ampicillin resistance marker to create this Golden Gate ready vector. Genes and barcode-specific landing pad sequences were cloned into the recall plasmid using the type IIS restriction sites. Barcode-specific landing pad arrays were generated by ordering phosphorylated complimentary oligo pairs, corresponding with the barcode sequence of interest, with specific overlaps that both direct assembly of the landing pad array and integration into the Recall plasmid. The landing pad arrays were ligated and gel extracted to ensure cloning with a fully assembled array. The fully assembled barcode landing pad was cloned into the BbsI site using standard restriction digest cloning. Mock Recall screens were used to assess efficiency via lineage specific expression of sfGFP. This reporter construct was assembled by cloning in a gBlock® encoding miniCMV-sfGFP into the BsaI site using Golden Gate Assembly (described below). Lineage-specific cell death was measured via barcode driven expression BAX and the hyper active mutant BAX D71A. gBlocks® encoding miniCMV-BAX and miniCMV-BAX D71A were cloned into the BsmBI sites using Golden Gate Assembly.

Mock Recall Screens

The mock screens were performed in 24 well plates. HEK293T cells were transfected at 60% confluence using 1.5 μl Lipofectamine™3000, 1 μl P3000™ Reagent, 150 ng of Recall plasmid and 500 ng of dCas9-VPR plasmid. Caco2 cells were transfected at 30% confluence and transfected using 1 μl Lipofectamine™LTX, 0.5 μl Plus™ Reagent, 250 ng Recall Plasmid, and 250 ng dCas9-VPR plasmid. MB-MDA-231 cells were transfected at 70% confluence using 1 μl Lipofectamine™LTX, 0.5 μl Plus™ Reagent, 250 ng Recall Plasmid, and 250 ng dCas9-VPR plasmid. Cells were analyzed for GFP expression via flow cytometry 48 hours post-transfection.

Lineage Isolation

For a standard, a range of HEK293T Bg-1 in barcode-gRNA library dilutions were plated in a 6 well plate with total cell number 360,000 per well. Two 10 cm plates were plated at 2.2 million cells for both a 1% and 0.1% Bg-1 lineage dilution for lineage isolation. The 6 well plates were transfected with 4.5 μl Lipofectamine™LTX, 2.25 μl Plus™ Reagent, 675 ng Recall Plasmid, and 1.575 μg dCas9-VPR plasmid per well. The 10 cm plates were transfected with 27.5 μl Lipofectamine™LTX, 13.75 μl Plus™ Reagent, 4.125 μg Recall Plasmid, and 9.625 μg dCas9-VPR plasmid per plate. Sorting gates were set using 0% Bg-1 as a standard. Isolated cells were set for and later harvested for genomic DNA.

Annexin V Red Assay

Caco2 were transfected at 30% confluence using 1 μl Lipofectamine™LTX, 0.5 μl Plus™ Reagent, 250 ng Recall Plasmid, and 250 ng dCas9-VPR plasmid. At time of transfection, 2.5 μl IncuCyte® Annexin V Red Reagent (Essen BioScience Cat #4641) was added to monitor apoptosis. Cells were monitored in the IncuCyte® for real time measurement of apoptotic cells in culture via fluorescent quantitation. Images were collected every 120 min and quantitation of apoptotic was performed using the IncuCyte® image analysis software.

### Example 3: Baar

Disclosed herein is a novel multi-tool barcoding method, Barcode Assisted Ancestral Recall (BAAR), that allows for both high-resolution lineage tracking and subsequent isolation of purified cell lineages for downstream analysis. Lineage tracing via barcoding is typically a destructive measurement; however, with the BAAR system there exists the ability to return to an earlier time point in the evolutionary trajectory and retrieve selected lineages of interest. The ability to concurrently track clonal fitness dynamics and generate lineage-specific genomic and transcriptomic data over longitudinal studies gives unprecedented insight into cancer adaptation and evolution.

Chemo-resistance is the major reason for therapy failure. One application of the BAAR platform is to perform ex vivo testing of tumor cells in order to stay “one step ahead” of emerging resistant clones. As an ex vivo patient-specific tool, tumor cells are labeled with a library (more than 10⁶ unique tags) of novel expressed barcodes, cultured as patient-derived organoids and treated with the same first-line treatment as patients. In multiple parallel samples, one can monitor the growth dynamics of the post-treatment population and determine which clones survive the treatment or may even have a growth advantage. Using BAAR, these resistant clones of interest can be purified from an untreated population and evaluated to identify appropriate second and third line treatments that target the resistant survivor cell population.

Downstream analyses of the resistant cell population of interest can include genomic and transcriptome analyses, drug library screening, metabolic analyses, and many other functional assays.

Lineage-specific expression of a fluorescent reporter by BAAR has been demonstrated. Error load has been quantified and low false positive/false negative rates were achieved. This platform has been deployed in a variety of cell types including HEK293T, CRC (Caco2), breast cancer (MB-MDA-231), lung adenocarcinoma (HCC827) and ovarian carcinoma (SKOV3). In order to translate this tool to a more clinical setting, its function with patient-derived tumor cells is validated. The power of the system can be tested to retrieve the resistant lineages following treatment with standard of care chemotherapeutic drugs.

Utilizing Cell Lineage Tracking and Isolation System BAAR Cultures of Patient-Derived Colorectal Carcinoma Cells.

Existing workflow is established for the generation of high diversity barcode-tagged cell populations in patient-derived cultures. As reference standards, the efficiency of cellular lineage tracking and retrieval is tested using a) a reference set of low diversity barcodes and then b) a reference barcode in the background of a high diversity library of ˜10⁶ barcodes.

Patient-Derived CRC Biospecimens.

From existing CRC biospecimens, 6 KRAS PDX models are evaluated using an organoid culture (PDO) technique. There is diversity in this selected group with regard to CMS and gene mutations.

Establishment of CRC Organoid Culture.

Briefly, each independent PDO is grown and expanded, embedded in extracellular matrix (ECM) gel (Matrigel, 50 μL) in 24-well plates by replenishing fresh complete medium (Advanced DMEM/F12, human epidermal growth factor (EGF), ROCK inhibitor, TGF-β inhibitor, and other supplements) containing conditioned medium (R-Spondin 3, and Noggin) until the average size of organoids reaches 600-700 μm.

Lineage Barcoding and Isolation.

A high diversity barcode library of greater than 10⁶ unique barcodes has been constructed. In this high-diversity background, small quantities of a known reference barcodes are added to the sample as a standard. The relative ratio of the reference barcode varies between 1% and 0.01% of the total. Barcodes are stably integrated into the host cell genome of CRC using lentiviral delivery at low MOI. Recall plasmids are constructed for the reference barcodes, transfected to cell populations, and both the GFP+ and GFP-fractions will be collected for barcode sequencing.

Demonstration of the Utility of BAAR for the Retrieval of Resistant Lineages Following Treatment with Standard of Care Chemotherapeutic Drugs.

PDO cultures are screened with a small set of compounds in clinical use for CRC. These include irinotecan, oxaliplatin and 5-FU, first and second-line chemotherapeutics for CRC treatment. PDOs are cultured with each agent alone (or vehicle) and in combination (oxaliplatin+5-FU). Resistant cell lineages are isolated from earlier time points in organoids and screened separately to identify potential rational drug combinations.

Organoid Culture.

PDOs are cultured as described above. 6 KRAS PDO models are screened using the organoid culture technique described above.

Barcode Labeling.

As validated above, a high diversity library of greater than 10⁶ unique promoter-barcode-gRNA DNA cassettes are stably integrated into the host cell genome of the CRC cells by lentiviral transduction. The cell population is transduced in single cell suspension at low MOI (0.1-0.2) to minimize the incorporation of multiple barcodes into a single cell. Cells are then plated in ECM for organoid culture according to our standard protocols.

Drug Sensitivity Assays of Organoids.

Organoids are screened in triplicate using drug concentrations ranging from 1 nM to 100 μM using serial dilution steps. PDO sensitivity to oxaliplatin, irinotecan and 5-FU, first line chemotherapeutics for CRC treatment, is tested. After PDOs are treated with single agents, they are exposed to oxaliplatin+5-FU combination (using ˜IC₂₅₋₃₀ for each drug). Cell viability is assayed using luminescence (CellTiter-Glo) quantified on a plate reader. Organoid cytotoxic responses are stratified as having minimal, moderate or high sensitivity. After 72 hours of drug exposure, PDOs are retrieved from the extracellular matrix and processed for BAAR code analysis to compare drug resistant clones to sensitive and untreated samples.

Lineage Dynamics and Purification of Resistant Cells.

Quantitative lineage frequency data for duplicate PDO is generated by Illumina HiSeq 4000 analysis of the barcode frequencies. For each PDO, the most abundant cellular lineage in the drug resistant population is isolated from parallel PDO cultures. This is accomplished as above, by transfecting a Recall plasmid specific to each barcode of interest and collecting the lineage-specific GFP+subpopulation by FACS.

Analysis and Drug Screening of Resistant Cells.

Purified resistant cell lineages are subcultured and sensitivity to oxaplatin, irinotecan, 5-FU is measured. To identify altered pathways that may be actionable targets in these populations, RNASeq are performed.

### REFERENCES

1. Greaves M. Evolutionary determinants of cancer. Cancer Disc. 2015; 5: 806-820.
2. Brock, A., Chang, H. & Huang, S. Non-genetic heterogeneity—a mutation-independent driving force for the somatic evolution of tumours. Nat Rev Genet 10, 336-342 (2009). PMID: 19337290.
3. Sharma, S. V. et al. A chromatin-mediated reversible drug-tolerant state in cancer cell subpopulations. Cell 141, 69-80 (2010). PMID: 20371346. PMC 2851638.
4. Huang, S. & Kauffman, S. How to escape the cancer attractor: rationale and limitations of multi-target drugs. Semin Cancer Biol 23, 270-278 (2013). PMID: 23792873. PMC.
5. Polyak, K. Tumor Heterogeneity Confounds and Illuminates: A case for Darwinian tumor evolution. Nat Med 20, 344-346 (2014). PMID: 24710378. PMC.
6. Archetti, M., Ferraro, D. A. & Christofori, G. Heterogeneity for IGF-II production maintained by public goods dynamics in neuroendocrine pancreatic cancer. Proc Natl Acad Sci USA 112, 1833-1838 (2015). PMID: 25624490. PMC 4330744.
7. Grosse-Wilde, A. et al. Stemness of the hybrid Epithelial/Mesenchymal State in Breast Cancer and Its Association with Poor Survival. PLoS One 10, e0126522 (2015). PMID: 26020648. PMC PMC4447403.
8. Cleary, A. S., Leonard, T. L., Gestl, S. A. & Gunther, E. J. Tumour cell heterogeneity maintained by cooperating subclones in Wnt-driven mammary cancers. Nature 508, 113-117 (2014). PMID: 24695311. PMC 4050741.
9. Quintana, E. et al. Phenotypic heterogeneity among tumorigenic melanoma cells from patients that is reversible and not hierarchically organized. Cancer Cell 18, 510-523 (2010). PMID: 21075313. PMC.

Pisco, A. O. et al. Non-Darwinian dynamics in therapy-induced cancer drug resistance. Nature communications 4, 2467 (2013). PMID: 24045430. PMC.
11. McGranahan N, Swanton C. Clonal Heterogeneity and Tumor Evolution: Past, Present, and the Future. Cell. 2017 9; 168(4):613-628.
12. Bhang H E, Ruddy D A, Krishnamurthy Radhakrishna V, Caushi J X, Zhao R, Hims M M, Singh A P, Kao I, Rakiec D, Shaw P, Balak M, Raza A, Ackley E, Keen N, Schlabach M R, Palmer M, Leary R J, Chiang D Y1, Sellers W R, Michor F, Cooke V G, Korn J M, Stegmeier F. (2015) Studying clonal dynamics in response to cancer therapy using high-complexity barcoding. Nat Med, 21(5):440-8. PMID: 25849130.
13. Hata A N, Niederst M J, Archibald H L, Gomez-Caraballo M, Siddiqui F M, Mulvey H E, Maruvka Y E, Ji F, Bhang H E, Krishnamurthy Radhakrishna V, Siravegna G, Hu H, Raoof S, Lockerman E, Kalsy A, Lee D, Keating C L, Ruddy D A, Damon L J, Crystal A S, Costa C, Piotrowska Z, Bardelli A, Iafrate A J, Sadreyev R I, Stegmeier F, Getz G, Sequist L V, Faber A C, Engelman J A. Tumor cells can follow distinct evolutionary paths to become resistant to epidermal growth factor receptor inhibition. Nat Med. 2016 March; 22(3):262-9. doi: 10.1038/nm.4040.
14. Levy S F, Blundell J R, Venkataram S, Petrov D A, Fisher D S, Sherlock G. Quantitative evolutionary dynamics using high-resolution lineage tracking. Nature. 2015 12; 519(7542):181-6. doi: 10.1038/nature14279.
15. Blundell, J R and Levy, S F. (2014) Beyond genome sequencing: Lineage tracking with barcodes to study the dynamics of evolution, infection, and cancer. Genomics 104 (2014) 417-430. PMID: 25260907.

1. A method of modulating expression of a gene of interest within a select population of cells comprising:
a. providing a population of cells;
b. providing a vehicle, plasmid, vector or recombinant virus, or equivalent thereof, capable of stably expressing a guide nucleic acid comprising randomized barcodes, thereby producing a population of barcoded cells;
c. allowing said barcoded cell to divide, thereby forming a barcoded progeny of cells;
d. saving an aliquot of cells after step b) or step c);
e. identifying the barcode in a lineage of interest from the barcoded progeny of cells;
f. reconstituting the aliquot of cells from step c) and transforming the reconstituted aliquot of cells to a transcriptional element comprising a transcriptional effector, the barcode of the lineage of interest, and a gene of interest;
g. utilizing the transcriptional effector to modulate expression of the gene of interest within the lineage of interest.

2. The method of claim 2, wherein the transcriptional effector is dCas9-VPR.

3. The method of claim 1, wherein the gene of interest is a reporter.

4. The method of claim 1, wherein cells are selected via cell sorting.

5. The method of claim 4, wherein cell sorting is done using single cell sorting, fluorescent activated cell sorting (FACS), physical cell manipulation, laser capture, or magnetic cell sorting.

6. The method of claim 1, wherein the barcode is created with a DNA construct.

7. The method of claim 6, wherein the DNA construct comprises a randomized barcoded crRNA segment upstream of a tracrRNA under control of a promoter.

8. The method of claim 1, wherein, prior to identifying the barcode in a lineage of interest, the cells are exposed to a candidate agent.

9. The method of claim 8, wherein the barcode in a lineage of interest is identified as being of interest based on an activity of the candidate agent.

10. The method of claim 9, wherein the activity is modulation of a given activity of the cell.

11. The method of claim 10, wherein the modulation is upregulation of a certain gene or genes.

12. The method of claim 11, wherein the modulation of downregulation of a certain gene or genes.

13. The method of claim 10, wherein the candidate agent causes apoptosis.

14. The method of claim 10, wherein the candidate agent causes cell multiplication.

15. The method of claim 10, wherein a plurality of barcoded cells are each treated with a different candidate agent.

16. The method of claim 10, wherein said candidate agent is selected from one or more of: a protein, a small molecule, an organic molecule, a carbohydrate, a polysaccharide, a polynucleotide, a polypeptide, and a lipid.

17. The method of claim 1, wherein the genome of the selected cells can be sequenced.

18. The method of claim 1, wherein after step g), cells in the lineage of interest can be identified and selected.

19. The method of claim 1, wherein the transcriptional element is a plasmid.

20. The method of claim 1, wherein the barcode of the lineage of interest is upstream the gene of interest.

21. The method of claim 1, wherein the guide nucleic acid is gRNA.

22-63. (canceled)
